SEQUENCE LISTING (7 sequences)

SEQ ID NO: 1 - DNA, 18 bp, Rattus rattus
  mRNA (1)..(18): Antisense oligonucleotide
tatcactcctggaagccc

SEQ ID NO: 2 - DNA, 18 bp, Rattus rattus
  mRNA (1)..(18): Antisense oligonucleotide
tctgagcactaaagctgg

SEQ ID NO: 3 - DNA, 18 bp, Rattus rattus
  mRNA (1)..(18): Antisense oligonucleotide
gtgatagtatgccgagca

SEQ ID NO: 4 - DNA, 18 bp, Rattus rattus
  mRNA (1)..(18): Antisense oligonucletotide
cgttacgtaagcctagga

SEQ ID NO: 5 - DNA, 18 bp, Rattus rattus
  mRNA (1)..(18): Antisense oligonucleotide
gtgtcggggtctccgggc

SEQ ID NO: 6 - DNA, 18 bp, Rattus rattus
  mRNA (1)..(18): Antisense oligonucleotide
catgtcctccaccttgga

SEQ ID NO: 7 - DNA, 18 bp, Rattus rattus
  mRNA (1)..(18): Antisense oligonucleotide
gatcaggcgtgcctcaaa

(19) United States
(12) Patent Application Publication
(10) Pub. No.: US 2002/0151513 A1
(43) Pub. Date: Oct. 17, 2002

(54) Localized oligonucleotide therapy for preventing restenosis

(76) Inventors: Martin G. Sirois, Montreal (CA); Elazer R. Edelman, Brookline, MA (US); Robert D. Rosenberg, Jamestown, RI (US); Michael Simons, Hanover, NH (US)

Correspondence Address: QUARLES & BRADY LLP, 411 E. WISCONSIN AVENUE, SUITE 2040, MILWAUKEE, WI, 53202-4497, US

(21) Appl. No.: 09/945,131

(22) Filed: Aug. 31, 2001

Related U.S. Application Data
(63) Continuation-in-part of application No. 09/241,561, filed on Feb. 1, 1999, now abandoned.

(51) Int. Cl. A61K 48/00; C07H 21/04

(52) U.S. Cl. 514/44; 536/23.2

(57) ABSTRACT

Antisense oligonucleotide gene therapy selective for the 5′ region of PDGFR-β subunit mRNA was used in attempt to prevent intimal thickening following rat carotid arterial injury. Sustained perivascular application of the antisense oligomers for 14 days reduced PDGFR-β protein overexpression and prevented neointima formation by 80%. Alternatively, a bolus of antisense oligomers reduced the PDGFR-β protein expression by at least 90% for at least 28 days. Specificity was verified by the absence of effects on the expression of a non-targeted gene PDGFR-α. These data demonstrated that antisense oligonucleotide sequences can effectively suppress a growth factor receptor, and the reduction of intimal hyperplasia after injury correlates with the extent to which these oligomers inhibited PDGFR-β protein expression. Advantageously, reduction of intimal hyperplasia was also accomplished with an almost completely restored endothelial function. Methods and materials useful for preventing restenosis are described and claimed.

### BACKGROUND OF THE INVENTION

This invention relates to a method of delivery of antisense oligonucleotide to a preselected locus in vivo, useful in the treatment of disease.

In the last several years, it has been demonstrated that oligonucleotides are capable of inhibiting the replication of certain viruses in tissue culture systems. For example, Zamecnik and Stephenson, Proc. Natl. Acad. Sci. U.S.A., 75: 280-284 (1978), showed oligonucleotide-mediated inhibition of virus replication in tissue culture, using Rous Sarcoma Virus. Zamecnik et al., Proc. Natl. Acad. Sci. U.S.A., 83: 4145-4146 (1986), demonstrated inhibition in tissue culture of the HTLV-III virus (now HIV-1) which is the etiological agent of AIDS. Oligonucleotides also have been used to suppress expression of selected non-viral genes by blocking translation of the protein encoded by the genes. Goodchild, et al., Arch. Biochem. Biophys., 264: 401-409 (1988) report that rabbit-globin synthesis can be inhibited by oligonucleotides in a cell-free system. Treatment with antisense c-myb has been shown to block proliferation of human myeloid leukemic cell lines in vitro. G. Anfossi, et al., Proc. Natl. Acad. Sci. USA, 86: 3379 (1989).

A drawback to this method is that oligonucleotides are subject to being degraded or inactivated by cellular endogenous nucleases. To counter this problem, some researchers have used modified oligonucleotides, e.g., having altered internucleotide linkages, in which the naturally occurring phosphodiester linkages have been replaced with another linkage. For example, Agrawal et al., Proc Natl. Acad. Sci. U.S.A., 85: 7079-7083 (1988) showed increased inhibition in tissue culture of HIV-1 using oligonucleotide phosphoramidates and phosphorothioates. Sarin et al., Proc. Natl. Acad, Sci. U.S.A., 85: 7448-7451 (1988) demonstrated increased inhibition of HIV-1 using oligonucleotide methylphosphonates. Agrawal et al., Proc. Natl. Acad. Sci. U.S.A., 86: 7790-7794 (1989) showed inhibition of HIV-1 replication in both early-infected and chronically infected cell cultures, using nucleotide sequence-specific oligonucleotide phosphorothioates. Leither et al., Proc. Natl. Acad. Sci U.S.A., 87; 3430-3434 (1990) report inhibition in tissue culture of influenza virus replication by oligonucleotide phosphorothioates.

Oligonucleotides having artificial linkages have been shown to be resistant to degradation in vivo. For example, Shaw et al., in Nucleic Acids Res., 19: 747-750 (1991), report that otherwise unmodified oligonucleotides become more resistant to nucleases in vivo when they are blocked at the 3′ end by certain capping structures and that uncapped oligonucleotide phosphorothioates are not degraded in vivo.

While antisense oligonucleotides have been shown to be capable of interfering selectively With protein synthesis, and significant progress has been made on improving their intracellular stability, the problem remains that oligonucleotides must reach their intended intracellular site of action in the body in order to be effective. Where the intended therapeutic effect is a systemic one, oligonucleotides may be administered systemically. However, when it is necessary or desirable to administer the oligonucleotide to a specific region within the body, systemic administration typically will be unsatisfactory. This is especially true when the target mRNA is present in normal cells as well as in the target tissue, and when antisense rRNA binding in normal cells induces unwanted physiological effects. Stated differently, the dosage of antisense oligonucleotide administered systemically that is sufficient to have the desired effect locally may be toxic to the patient. An example of a treatment strategy which could greatly benefit from development of a method of limiting the effect of antisense oligonucleotide to a target tissue is the inhibition of smooth muscle cell proliferation which leads to restenosis following vascular trauma.

Smooth muscle cell proliferation is a poorly understood process that plays a major role in a number of pathological states including atherosclerosis and hypertension. It is the leading cause of long-term failure of coronary and peripheral angioplasty as well as of coronary bypass grafts.

Vascular smooth muscle cells in adult animals display a well defined phenotype characterized by an abundance of contractile proteins primarily smooth muscle actin and myosins, as reviewed by S. M. Schwartz, G. R. Campbell, J. H. Campbell, Circ. Res., 58: 427 (1986), and a distinct lack of rough endoplasmic reticulum. When subjected to injury in vivo or placed in an in vitro cell culture, adult smooth muscle cells (SMC) undergo a distinct phenotypic change and lose their “differentiated” state. The cells acquire large amounts of endoplasmic reticulum and gain actively synthesizing extracellular matrix, and they begin expressing a number of new proteins.

U.S. Pat. No. 5,593,974 describes the therapeutic effect of antisense oligonucleotides against the PCNA, c-myb and NMMHC mRNAs, when locally administered in damaged vascular tissue. It is inferred to, in this reference, that smooth muscle growth is stimulated by PDGF (platelet-derived growth factor). Further, in the patent publication WO 93/08845, it is also mentioned that antisense could be made against the messengers of PDGF and its vascular receptor. These two references do not teach that antisense oligonucleotides to these molecules would effectively prevent restenosis.

It is now widely accepted that within the first 2 days following vascular injury damaged and dying medial vascular smooth muscle cells (vSMC) release growth promoters such as bFGF. This induces vSMC proliferation for the next 3-5 days, delineating the first wave of the vascular healing process (1-3). The second and third waves rely on migration of medial vSMC and their proliferation within the neointima (4) It is thought that half of the migrating vSMC will undergo 3 rounds of cell cycle proliferation in the intima, ultimately representing nearly 90% of the final cell count in the neointima. The other half of the migrating vSMC do not divide, and account for the remaining 10% of the intimal cell count (1). vSMC are observed within the neointima as soon as 3 days after the injury. Their number peaks within 2 weeks of injury and remains relatively constant for up to 1 year (5). Several molecules such as angiotensin 11, TGF-β, bFGF and PDGF-BB might act as vSMC chemotactic factors during the second wave of cellular events (4). PDGF-BB has received particular attention because it is both mitogenic for cultured vSMC through activation of either PDGF receptors (PDGFR-αα or PDGFR-ββ), and chemotactic through the activation of PDGFR-ββ (6). In vivo, however, PDGF-BB acts predominantly as a chemotactic factor on vSMC. Injection of this growth factor increased vSMC migration by 10-20 fold, but proliferation by no more than 2 fold (7), and polyclonal anti-PDGF antibodies blocked the migration of vSMC migration, but not their proliferation (8). It is therefore, reasonable to postulate that PDGF-BB plays a critical role in intimal thickening during the first 2 weeks after a vascular lesion.

PDGFR-β subunit is specifically expressed in mesenchymal cells, such as vSMC and fibroblasts (22). Basal expression in the medial vSMC of the normal artery increases within days of injury (23). What is not known is whether PDGF receptor expression is directly related to the extent of neointimal hyperplasia. Antisense oligonucleotide gene therapy enables us to examine this question (10-17). Antisense oligonucleotide sequences hybridize (18-20) with targeted mRNA or gene regions at ribosomic or nuclear sites preventing mRNA translation into protein (21). To date, antisense oligonucleotides directed against growth-regulatory or cell-cycle genes (c-myb, c-myc, PCNA, cdc2, cdk2) involved in vSMC proliferation after injury have successfully altered intimal hyperplasia (10-17). Yet, to the best of our knowledge no one has used antisense sequences to prevent the expression of chemotactic proteins or their receptors. We examined these issues by examining the effect of antisense phosphorothioate-oligodeoxyribonucleotide sequences complementary to PDGFR-β mRNA on PDGFR-β protein expression and intimal thickening after vascular injury. The sustained release of PDGFR-β mRNA antisense oligonucleotide reduced PDGFR-β protein expression and intimal thickening in injured rat carotid arteries in an exponentially correlative fashion. Thus, myointimal proliferation depends on both PDGFR-β subunit overexpression and its activation by platelet-derived PDGF-BB. Removal of either one of these two elements can suppress neointima formation.

We further investigated whether a single endovascular delivery of AS PDGFR-β would be sufficient to reduce intimal hyperplasia by limiting either VSMC migration or proliferation. We also investigated the possibility that inhibition of PDGFR-β overexpression would favor endothelial regrowth and the return of vasomotor activity.

### SUMMARY OF THE INVENTION

The present invention relates to a method for inhibiting translation or transcription of a target nucleic acid sequence preferentially at a locus in vivo. The invention involves application directly to the target tissue through a surgical or catheterization procedure of specific oligonucleotides having a nucleotide sequence complementary to at least a portion of the target nucleic acid, i.e., antisense oligonucleotides. The oligonucleotides are preferably antisense sequences specific for the messenger RNA (mRNA) transcribed from the gene whose expression is to be inhibited. The antisense oligonucleotides hybridize with the target mRNA thereby preventing its translation into the encoded protein. Thus, the present method prevents the protein encoded by a selected gene from being expressed Furthermore, animal experiments have demonstrated dramatic local therapeutic effects in vivo.

The present oligonucleotides preferably are modified to render them resistant to degradation and/or extension by cellular nucleases or other enzymes present in vivo. This can be accomplished by methods known in the art, e.g., by incorporating one or more internal artificial internucleotide linkages, such as replacing the phosphate in the linkage with sulfur, and/or by blocking the 3′ end of the oligonucleotide with capping structures. Oligonucleotides of the present invention are preferably between about 14 and 38 nucleotides in length, more preferably between 15 and 30 nucleotides.

The oligonucleotides are applied locally in order to suppress expression of the protein of choice in a circumscribed area. In a preferred embodiment, the antisense oligonucleotide is applied to the surface of the tissue at the locus disposed within a biocompatible matrix or carrier. The matrix or carrier can be a hylrogel material such as a poly(propylene oxide-ethylene oxide) gel, e.g., one which is liquid at or below room temperature, and is a gel at body temperature and above. In this embodiment, the oligonucleotides are mixed with the hydrogel material, and the mixture is applied to the desired location during surgery or by catheter. The oligonucleotides also can be applied in solution by liquefying the gel, i.e., by cooling, and are retained at the area of application as the gel solidifies. Carriers which can be used also include, for example, liposomes, microcapsules, erythrocytes and the like. The oligonucleotides also can be applied locally by direct injection, can be released from devices such as implanted stents or catheters, or delivered directly to the site by an infusion pump.

The methods of the present invention are useful in inhibiting the expression of protein encoding genes, as well as regulating non-encoding DNA such as regulatory sequences. Since the antisense oligonucleotides are delivered to a specific defined locus, they can be used in viva when systemic administration is not possible. For example, systemically administered oligonucleoticies may be inactivated by endonucleases rendering them ineffective before they reach their targets. Large closes of the oligonucleotide may be necessary for successful systemic treatment systemically, which may have harmful or toxic effects on the patient. The present method provides a means for treating a large number of specific disorders using oligonucleotide therapy by delivering an antisense sequence to the specific location where it is needed.

The elucidation of molecular mechanisms of vascular cell biology has markedly influenced our thinking on the pathophysiology of vascular disease. Antisense oligonucleotide gene therapy have helped identify proteins critical to cell cycle progression and proliferation and possible therapeutic strategies to combat human disease. This approach, however, has not yet been employed to examine the contribution of chemotactic proteins and/or their receptors. PDGF-BB released from activated platelets adherent to subendothelial connective tissue is one of the principal smooth muscle cell chemotactic factor.

A series of experiments were performed to assess: 1) the capacity of antisense oligonucleotides to reduce PDGFR-P subunit expression and 2) the contribution of PDGFR-β subunit in neointimal formation. Sustained, direct and local perivascular administration of two different antisense oligonucleotide sequences complementary to PDGFR-β subunit mRNA almost completely abolished the expression of PDGFR-β protein in the intima and media of injured carotid arteries, and decreased neointima formation by 80 and 60% respectively. Furthermore, neointima formation correlated precisely with PDGFR-β subunit expression in an exponential fashion.

Thus, myointimal proliferation depends on both PDGFR-β subunit overexpression and its activation by platelet-derived PDGF-BB. Removal of either one of these two elements can suppress neointima formation.

In another complementary study, we have observed that a bolus of antisense PDGFR-β delivered into injured rat carotid arteries reduced PDGFR-β protein overexpression by >90% from day 3 to 28 after injury. At day 28 after injury, compared with injured untreated carotids, treatment with antisense PDGFR-β reduced intimal hyperplasia by 58% and medial VSMC migration by 49% and improved vascular reendothelialization by 100% and vascular reactivity (EC₅₀) to acetylcholine by 5-fold.

Therefore, a single-bolus luminal delivery of antisense PDGFR-β to injured rat carotids reduced intimal hyperplasia, improved the reendothelialization process, and led to the recovery of endothelium-dependent regulation of vascular tone.

### DETAILED DESCRIPTION OF THE INVENTION

A method for inhibiting expression of protein encoding genes using antisense oligonucleotides is described. The method is based on the localized application of the oligonucleotides to a specific site in vivo. The oligonucleotides preferably are applied directly to the target tissue in mixture with an implant or gel, or by direct injection or infusion. In one aspect, the oligonucleotides are treated to render them resistant in vivo to degradation or alteration by endogenous enzymes.

The Oligonucleotides

The therapeutic approach using antisense oligonucleotides is based on the principle that the function of a gene can be disrupted by preventing transcription of the gene or translation of the protein encoded by that gene. This can be accomplished by providing an appropriate length oligonucleotide which is complementary to at least a portion of the messenger RNA (mRNA) transcribed from the gene. The antisense strand hybridizes with the mRNA and targets the mRNA for destruction thereby preventing ribosomal translation, and subsequent protein synthesis.

The specificity of antisense oligonucleotides arises from the formation of Watson-Crick base pairing between the heterocyclic bases of the oligonucleotide and complementary bases on the target nucleic acid. For example, a nucleotide sequence sixteen nucleotides in length will be expected to occur randomly at about every 4¹⁶, or 4×10⁹ nucleotides. Accordingly, such a sequence is expected to occur only once in the human genome. In contrasts a nucleotide sequence of ten nucleotides in length would occur randomly at about every 4¹⁰ or 1×10⁶ nucleotides. Such a sequence might be present thousands of times in the human genome Consequently, oligonucleotides of greater length are more specific than oligonucleotides of lesser length and are less likely to induce toxic complications that might result from unwanted hybridization. Therefore, oligonucleotides of the present invention are preferably at least 14 nucleotide bases in length. Oligonucleotides having from about 14 to about 38 bases are preferred, most preferably from about 15 to 30 bases.

The oligonucleotide sequence is selected based on analysis of the sequence of the gene to be inhibited. The gene sequence can be determined, for example, by isolation and sequencing, or if known, through the literature. The sequence of the oligonucleotide is an “antisense” sequence, that is, having a sequence complementary to the coding strand of the molecule. Thus, the sequence of the oligonucleotide is substantially identical to at least a portion of the gene sequence, and is complementary to the mRNA sequence transcribed from the gene. The oligonucleotide therapy can be used to inhibit expression of genes from viruses or other microorganisms that are essential to infection or replication, genes encoding proteins involved in a disease process, or regulatory sequences controlling the expression of proteins involved in disease or other disorder, such as an autoimmune disorder or cardiovascular disease.

Oligonucleotides useful in the present invention can be synthesized by any art-recognized technique for nucleic acid synthesis. See, for example, Agrawal and Goodchild, Tetrahedron Letters, 28: 3539 (1987), Nielsen, et al., Tetrahedron Letters, 29: 2911 (1988): Jager et al., Biochemistry, 27: 7237 (1988); Uznanski et al., Tetrahedron Letters, 28: 3401 (1987): Bannwarth, Helv. Chim. Acta., 71:1517 (1988); Crosstick and Vyle, Tetrahedron Letters, 30: 4693 (1989); Agrawal, et al., Proc. Natl. Acad. Sci. USA, 87: 1401-1405 (1990), the teachings of which are incorporated herein by reference. Other methods for synthesis or production also are possible. In a preferred embodiment the oligonucleotide is a deoxyribonucleic acid (DNA), although ribonucleic acid (RNA) sequences may also be synthesized and applied.

The oligonucleotides useful in the invention preferably are designed to resist degradation by endogenous nucleolytic enzymes. In vivo degradation of oligonucleotides produces oligonucleotide breakdown products of reduced length. Such breakdown products are more likely to engage in non-specific hybridization and are less likely to be effective, relative to their full-length counterparts. Thus, it is desirable to use oligonucleotides that are resistant to degradation in the body and which are able to reach the targeted cells. The present oligonucleotides can be rendered more resistant to degradation in vivo by substituting one or more internal artificial internucleotide linkages for the native phosphodiester linkages, for example, by replacing phosphate with sulfur in the linkage. Examples of linkages that may be used include phosphorothioates, methylphosphonate, sulfone, sulfate, ketyl, phosphorodithioates, various phosphoramidates, phosphate esters, bridged phosphorothioates and bridged phosphoramidates. Such examples are illustrative, rather than limiting, since other internucleotide linkages are known in the art. See, e.g., Cohen, Trends in Biotechnology (1990). The synthesis of oligonucleotides having one or more of these linkages substituted for the phosphodiester internucleotide linkages is well known in the art, including synthetic pathways for producing oligonucleotides having mixed internucleotide linkages.

Methods of Application of the Oligonucleotides

In accordance with the invention, the inherent binding specificity of antisense oligonucleotides characteristic of base pairing is enhanced by limiting the availability of the antisense compound to its intended focus in vivo, permitting lower dosages to be used and minimizing systemic effects. Thus, oligonucleotides are applied locally to achieve the desired effect. The concentration of the oligonucleotides at the desired locus is much higher than if the oligonucleotides were administered systemically, and the therapeutic effect can be achieved using a significantly lower total amount. The local high concentration of oligonucleotides enhances penetration of the targeted cells and effectively blocks translation of the target nucleic acid sequences.

The oligonucleotides can be delivered to the locus by any means appropriate for localized administration of a drug. For example, a solution of the oligonucleotides can be injected directly to the site or can be delivered by infusion using an infusion pump. The oligonucleotides also can be incorporated into an implantable device which when placed at the desired site, permits the oligonucleotides to be released into the surrounding locus.

The oligonucleotides can be administered by means of numerous implants that are commercially available or described in the scientific literature, including liposomes, microcapsules and implantable devices.

The oligonucleotides may be administered via a hydrogel material as well. The hydrogel is noninflammatory and biodegradable. Many such materials now are known, including those mode from natural and synthetic polymers. In a preferred embodiment, the method exploits a hydrogen which is liquid below body temperature but gels to form a shape-retaining semisolid hydrogel at or near body temperature. Preferred hydrogel are polymers of ethylene oxide-propylene oxide repeating units. The properties of the polymer are dependent on the molecular weight of the polymer and the relative percentage of polyethylene oxide and polypropylene oxide in the polymer. Preferred hydrogels contain from about 10 to about 80% by weight ethylene oxide and from about 20 to about 90% by weight propylene oxide. A particularly preferred hydrogel contains about 70% polyethylene oxide and 30% polypropylene oxide. Hydrogels which can be used are available, for example, from BASF Corp, Parsippany, N.J., under the tradename Pluronic®.

In this embodiment, the hydrogel is cooled to a liquid state and the oligonucleotides are admixed into the liquid to a concentration of about 1 mg oligonucleotide per gram of hydrogel. The resulting mixture then is applied onto the surface to be treated, e.g., by spraying or painting during surgery or using a catheter or endoscopic procedures. As the polymer warms, it solidifies to form a gel, and the oligonucleotides diffuse out of the gel into the surrounding cells over a period of time defined by the exact composition of the gel.

Implants made of biodegradable materials such as polyanhydrides, polyorthoesters, polylactic acid and polyglycolic acid and copolymers thereof, collagen, and protein polymers, or non-biodegradable materials such as ethylenevinyl acetate (EVAc), polyvinyl acetate, ethylene vinyl alcohol, and derivatives thereof can be used to locally deliver the oligonucleotides. The oligonucleotides can be incorporated into the material as it is polymerized or solidified, using melt or solvent evaporation techniques, or mechanically mixed with the material. In one embodiment, the oligonucleotides are mixed into or applied onto coatings for implantable devices such as dextran coated silica beads, stents, or catheters.

As described in the following examples, the dose of oligonucleotides is dependent on the size of the oligonucleotides and the purpose for which is it administered. In general, the range is calculated based on the surface area of tissue To be treated. The effective dose of oligonucleotide is somewhat dependent on the length and chemical composition of the oligonucleotide but is generally in the range of about 30 to 3000 μg per square centimeter of tissue surface area. Based on calculations using the application of antisense myb in a hydrogen to blood vessel that has been injured by balloon angioplasty in a rat model, a dose of about 320 μg oligonucleotide applied to one square centimeter of tissue was effective in suppressing expression of the c-myb gene product.

The oligonucleotides may be administered to the patient systemically for both therapeutic and prophylactic purposes. For example, antisense oligonucleotides specific for PDGFR-β may be administered to a patient who is at risk for restenosis due to angioplasty or other procedure. The oligonucleotides may be administered by any effective method, for example, parenterally (e.g., intravenously, subcutaneously, intramuscularly or by oral, nasal or other means which permit the oligonucleotides to access and circulate in the patient's bloodstream.

Oligonucleotides administered systemically preferably are given in addition to locally administered oligonucleotides, but also have utility in the absence of local administration. A dosage in the range of from about 0.1 to about 10 grams per administration to an adult human generally will be effective for this purpose.

Therapeutic Applications

The method of the present invention can be used to treat a variety of disorders which are linked to or based on expression of a protein by a gene. The method is particularly useful for treating vascular disorders, particularly vascular restenosis. The following non-limiting examples demonstrate use of antisense oligonucleotides to prevent or very significantly inhibit restenosis following vascular injury Such as is induced by balloon angioplasty procedures. This has been already accomplished by using antisense, delivered locally, to inhibit expression of genes encoding proteins determined to be involved in vascular restenosis, including c-myb, non-muscle myosin heavy chain (NMMHC) and proliferative cellular nuclear antigen (PCNA). Particularly, this invention describes the use of antisense oligonucleotides against the messenger RNA molecules encoding the psubunit of the receptor for platelet-derived growth factor (PDGFR-β).

Expression of specific genes in specific tissues may be suppressed by oligonucleotides having a nucleotide sequence complementary to the mRNA transcript of the target gene. PDGFR-β protein appears to be critically involved in the initiation of migration and/or proliferation of smooth muscle cells. The inhibition of the production of this protein by antisense oligonucleotides offers a means for treating post-angioplasty restenosis and chronic processes such as atherosclerosis, hypertension, primary pulmonary hypertension. and proliferative glomerulonephritis, which involve proliferation of smooth muscle cells.

Illustrative of other conditions which may be treated with the present method are pulmonary disorders such as acute respiratory distress syndrome, idiopathic pulmonary fibrosis, emphysema, and primary pulmonary hypertension. These conditions may be treated, for example, by locally delivering appropriate antisense incorporated in an aerosol by inhaler. These disorders are induced by a complex overlapping series of pathologic events which take place in the alveolus (air side), the underlying basement membrane and smooth muscle cells, and the adjacent enclothelial cell surface (blood side). It is thought that the alveolar macrophage recognizes specific antigens via the T cell receptor, become activated and elaborates a variety of substances such as PDGF which recruit white blood cells as well as stimulate fibroblasts. White cells release proteases which gradually overwhelm the existing antiproteases and damage alveolar phneumocytes; fibroblasts secrete extracellular matrix which induce fibrosis. Selected growth factors such as PDGF and the subsequent decrease in blood oxygen, which is secondary to damage to the alveolar membrane, induce smooth muscle growth. This constricts the microvascular blood vessels and further decreases blood flow to the lung. This further decreases the transport of oxygen into the blood. The molecular events outlined above also induce activation of the microvascular endothelial cell surface with the appearance of selectins and integrins as well as the appearance of tissue factor which initiates blood coagulation. These selectin and integrin surface receptors allow white blood cells to adhere to microvascular endothelial cells and release proteases as well as other molecules which damage these cells and allow fluid to accumulate within the alveolus. The above events also trigger microvascular thrombosis with closure of blood vessels. The end result of this process is to further impede oxygen exchange.

Antisense oligonucleotides, locally delivered to the alveolar/microvascular area, could be directed against the following targets to intervene in the pathology outlined above, since the cDNA sequences of all of the targets selected are known. Thus, antisense oligonucleotides specific for mRNA transcribed from the genes would inhibit production of the alveolar macrophage T cell receptor to prevent initiation of the above events; inhibit product of a protein to prevent activation of alveolar white cells, or inhibit production of elastase to prevent destruction of alveolar membrane: inhibit production of PDGF to prevent recruitment of white cells or resultant fibrosis; inhibit production of c-myb to suppress SMC proliferation; inhibit production of p-selectin or e-selectin or various integrins to prevent adhesion of blood white cells to pulmonary microvascular endothelial cells; or inhibit the production of tissue factor and PAI-1 to suppress microvascular thrombosis.

As additional examples, Tissue Factor (TF) is required for coagulation system activation. Local application of antisense targeting the mRNA or DNA of a segment of TF in the area of clot formation can prevent additional coagulation. This therapy can be employed as an adjunct to or as a substitute for systemic anticoagulant therapy or after fibrinolytic therapy, thereby avoiding systemic side effects.

Plasminogen activator inhibitor (PAI-1) is known to reduce the local level of tissue plasminogen activator (TPA). The human cDNA sequence for PAI-1 is known. Local application of antisense targeting the mRNA or DNA of PAI-1 should permit a buildup of TPA in the targeted area. This may result in sufficient TPA production to naturally lyse the clot without systemic side effects.

A combination of antisense-TF and antisense-PAI-1 may be utilized to maximize the efficacy of treatment of several disorders, including local post thrombolytic therapy and preventative post-angioplasty treatment.

Many other vascular diseases can be treated in a manner similar to that described above by identifying the target DNA or mRNA sequence. The treatment of diseases which could benefit using antisense therapy include, for example, myocardial infarction, peripheral muscular disease and peripheral angioplasty, thrombophlebitis, cerebro-vascular disease (e.g., stroke, embolism), vasculitis (e.g., temporal ateritis) angina and Budd-Chiari Syndrome.

This method can be used against a variety of targets in addition to those detailed above. For example, DNA or mRNA encoding the following proteins could be used as target sequences: growth factors and receptors, including: PDGF-AA, PDGF-AB, PDGF-BB, PDGF-alpha Receptor, PDGF-beta Receptor.

This invention will be described hereinbelow by reference to the following preferred embodiments and appended figures which purpose is to illustrate rather than to limit its scope.

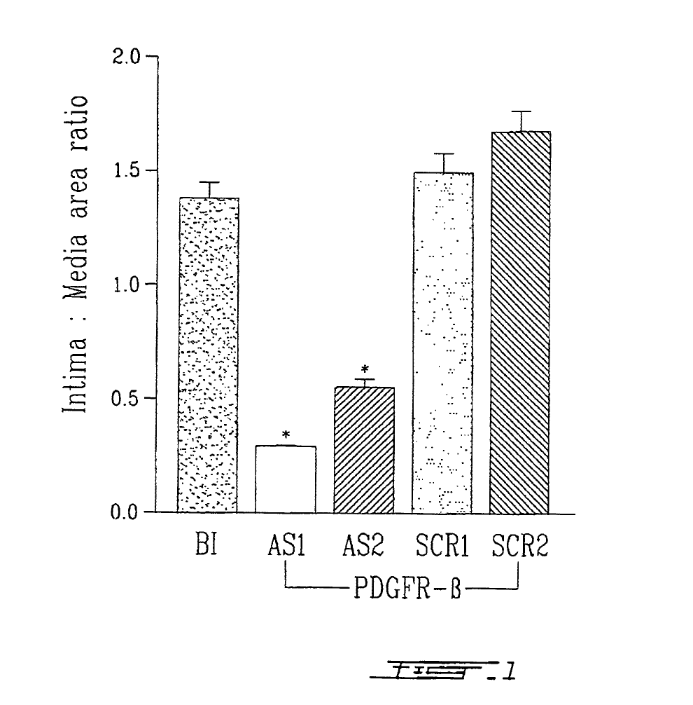

FIG. 1: Effects of mRNA PDGFR-β subunit antisense oligonucleotides on neointima formation: Following balloon denuding carotid arterial injury antisense oligonucleotide sequences corresponding to the fragment 4-21 (AS1) or 22-39 (AS2) or scrambled oligonucleotide of fragment 421 (SCR1) or 22-39 (SCR2) of 5′-region of PDGFR-β subunit mRNA were released into the perivascular space of injured vessels from implanted EVAc matrices. The rats were sacrificed 14 days later and the extent of neointimal hyperplasia expressed as the mean intima:media area ratio ±SE from 5-6 animals per group. *P<0.001 as compared to normal rats subject to balloon injury (BI).

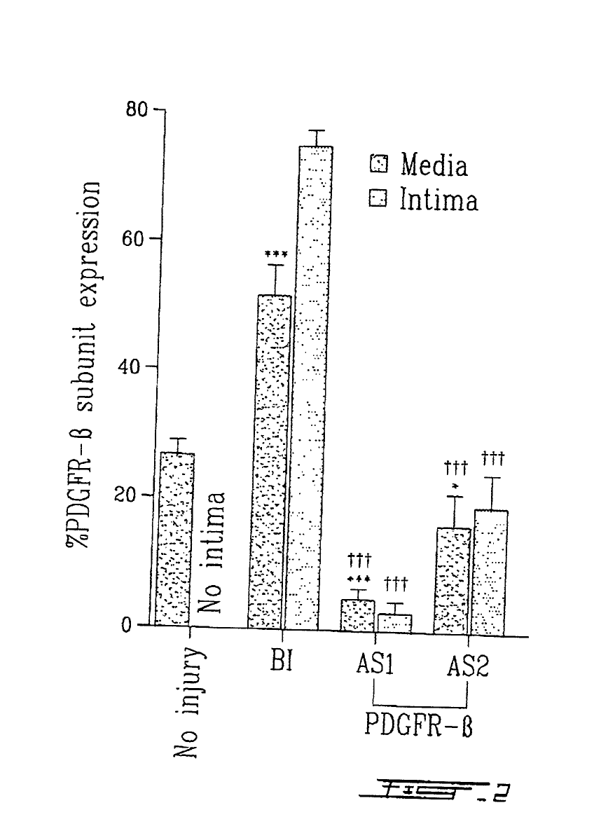

FIG. 2: Quantitative assessment of antisense oligonucleotide regulation of PDGFR-β subunit expression in injured carotid arteries: In the absence of injury (No injury) basal expression of PDGFR-P subunit reached 26.5±2.5% of all medial cells. Balloon denuding injury (BI) led to overexpression of PDGFR-β in both the media (black bars) and neointima (stippled bars). Both antisense oligonucleotide sequences (AS1 and AS2) to PDGFR-β subunit mRNA reduced the PDGFR-β subunit expression 14 days after the vascular injury. *P<0.05 and ***P<0.001 as compared to noninjured rats (No injury), and †††P<0.001 as compared to normal rats subject to balloon arterial denudation (BI).

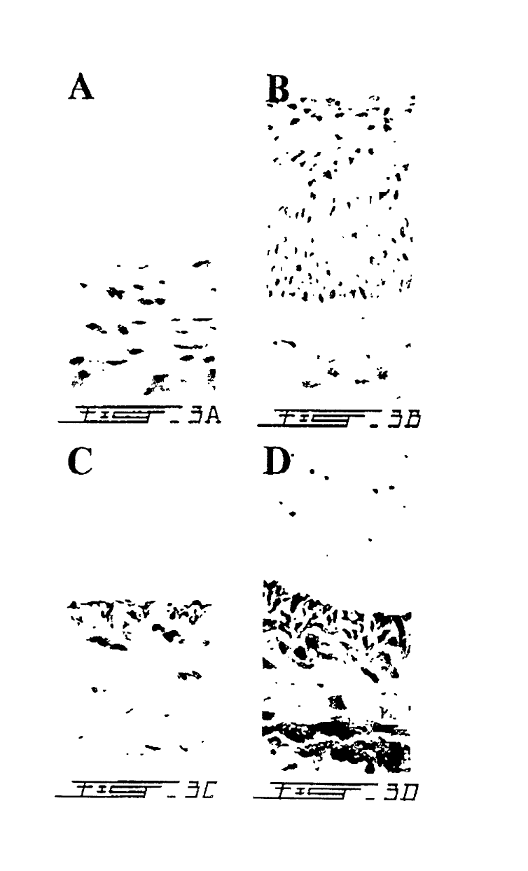

FIG. 3: Antisense oligonucleotide regulation of PDGFR-β subunit expression on representative cross sections of injured carotid arteries: In the absence of injury (A) basal expression of PDGFR-β subunit reached 26.5±2.5% of all medial cells. Balloon denuding injury led to overexpression in both the media and neointima (B). Both antisense oligonucleotide sequences complementary to PDGFR-β subunit mRNA reduced receptor subunit expression 14 days after the vascular injury (C-D), magnification (400×).

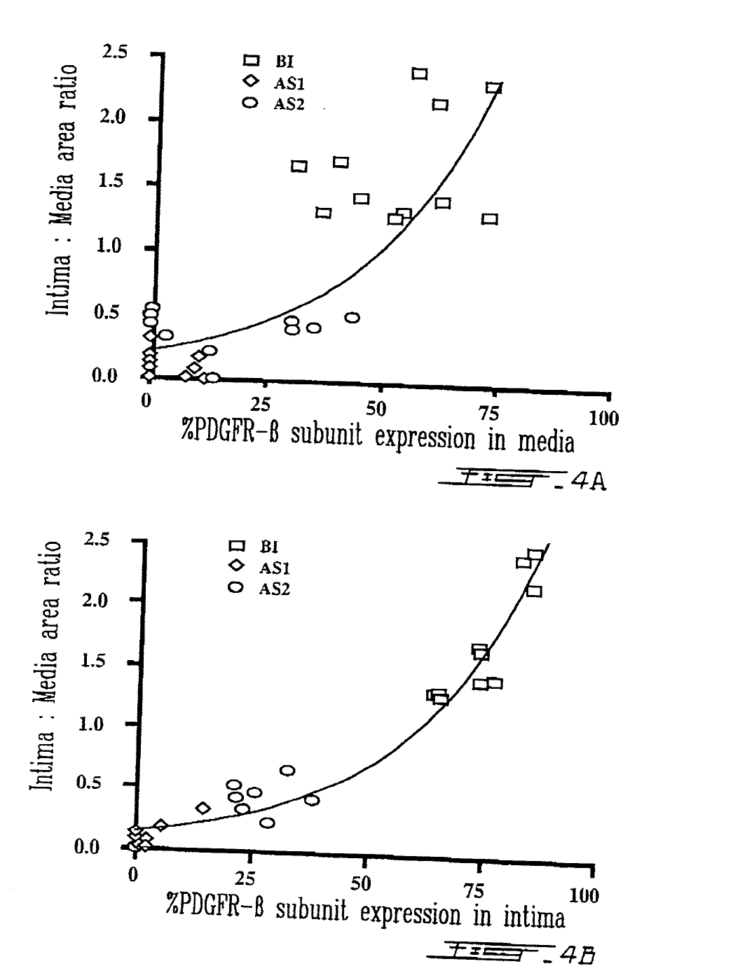

FIG. 4: Correlation of antisense regulation of PDGFR-β subunit expression and neointimal hyperplasia in injured carotid arteries: The upper and lower panels show respectively the expression of PDGFR-β subunit in the media and the intima versus the intima.media area ratio 14 days after balloon carotid arterial injury. Data was obtained from rats subject to balloon injury (BI) but not to antisense oligonucleotide sequences treatment ( ), and from rats that were treated either with AS1-PDGFR-β (⋄) or AS2-PDGFR-β (τ) Exponential fits were obtained in both cases.

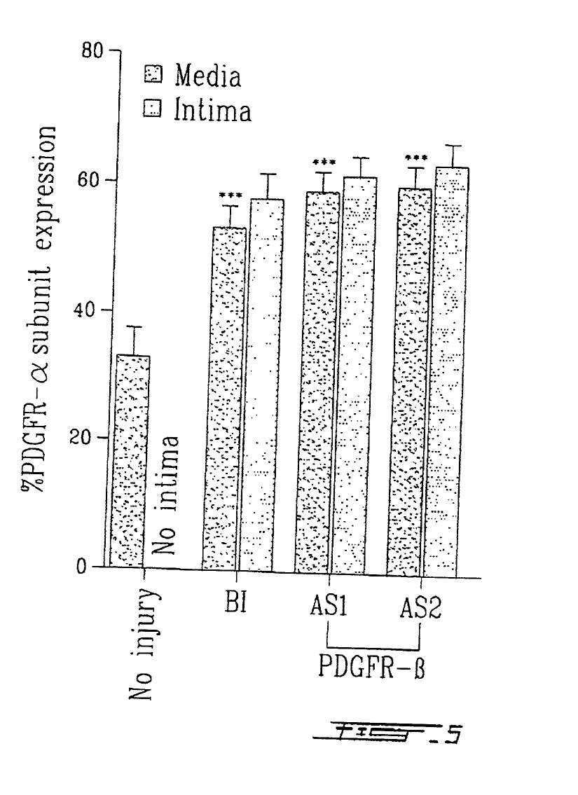

FIG. 5: Quantitative assessment of antisense oligonucleotide regulation of PDGFR subunit expression in injured carotid arteries: In the absence of injury (No injury) basal expression of PDGFR-α subunit reached 32.8±4.6% of all medial cells. Balloon denuding injury (BI) led to overexpression of PDGFR in both the media (black bars) and neointima (doted bars). Both antisense oligonucleotide sequences (AS1 and AS2) to PDGFR-P subunit mRNA did not reduce the PDGFR-α subunit expression 14 days after the vascular injury. ***P<0.001 as compared to noninjured rats (No injury).

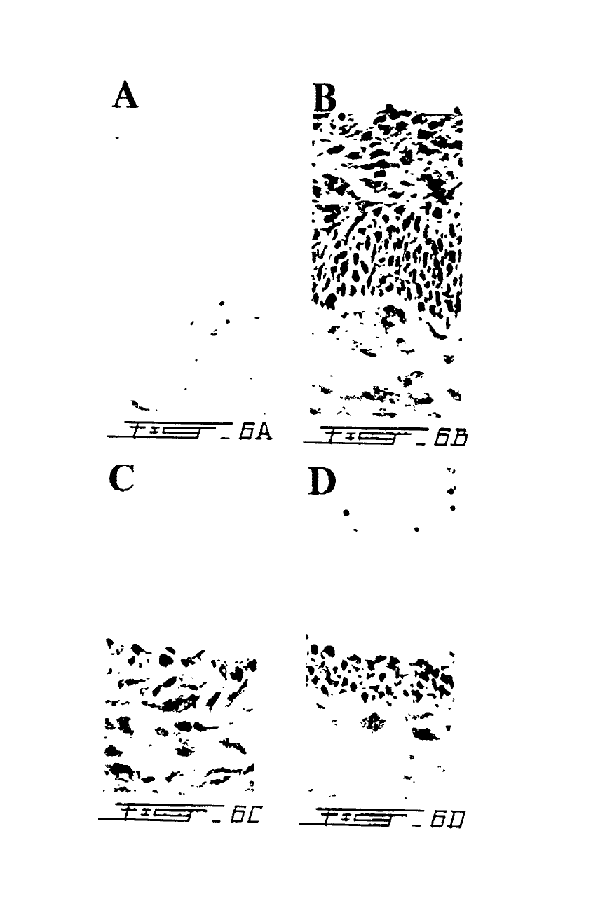

FIG. 6: Antisense oligonucleotide regulation of PDGFR-α subunit expression on representative cross sections of injured carotid arteries: In the absence of injury (A) basal expression of PDGFR-α subunit reached 32.8±4.6% of all media) cells. Balloon denuding injury led to overexpression of PDGFR-α in both the media and neointima (B). Neither of the antisense oligonucleotide sequences complementary to the PDGFR-β subunit mRNA reduced PDGFR-α subunit expression 14 days after the vascular injury (C-D), magnification (400×).

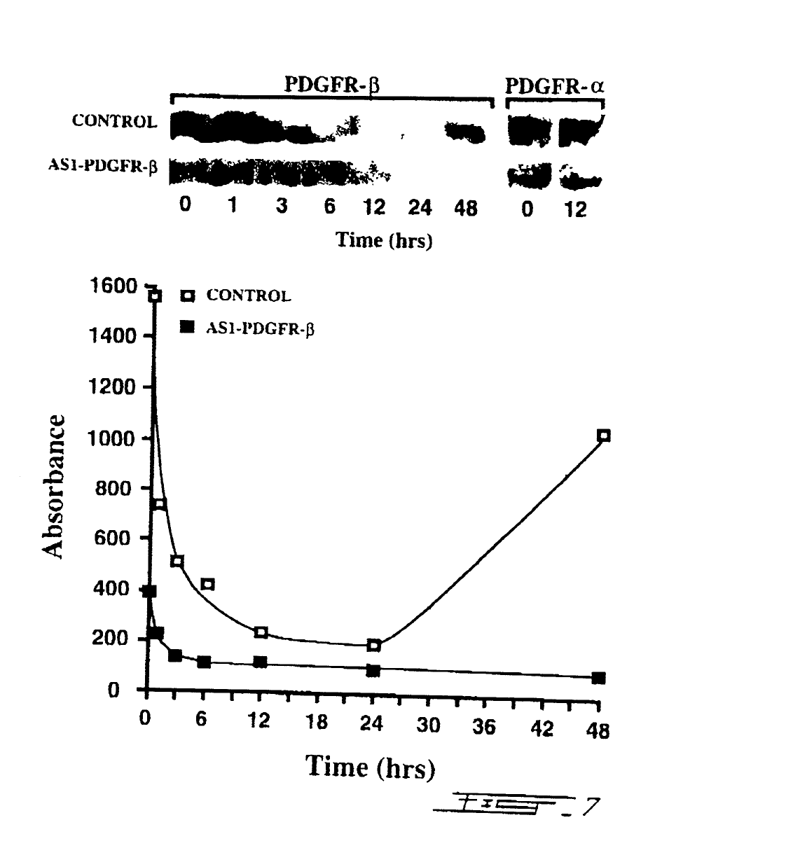

FIG. 7: Antisense regulation of PDGFR-β and PDGFR-α subunit expression on cultured vascular smooth muscle cells: Quiescent confluent rat vSMC were stimulated with 10 ng/ml of PDGF-BB and total proteins collected in Laemmli buffer 0, 1, 3, 6, 12, 24 and 48 hr later. One group of control cells was left without additional therapy (open squares), while an identical cohort treated with 20 μM AS1-PDGFR-β oligonucleotide 48 hrs, 24 hrs and immediately before PDGF-BB exposure (filled squares). Total protein (30 μg/lane) was applied on SDS-PAGE under reducing conditions, PDGFR-β and a protein expression were revealed by Western blot electrophoresis and immunohistochemistry, and quantified by image densitometry.

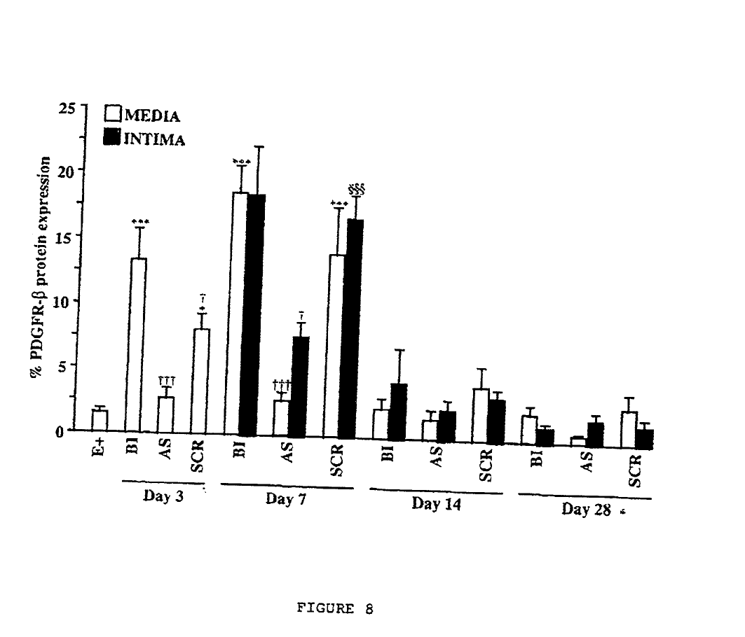

FIG. 8. Quantification of VSMCs expressing PDGFR-β protein. Baseline expression of PDGFR-β protein in media of uninjured carotid arteries (E+). Denuding BI led to PDGFR-13 protein overexpression (up to day 7) in BI and SCR-treated vessels and returned to basal level. AS-PDGFR-β reduced medial and intimal expression of PDGFR-β compared with BI. n=4 to 11 animals per treatment. One symbol, P<0.05; 2 symbols, P<0.01; 3 symbols, P<0.001 vs E+, tvs Bi, §vs AS.

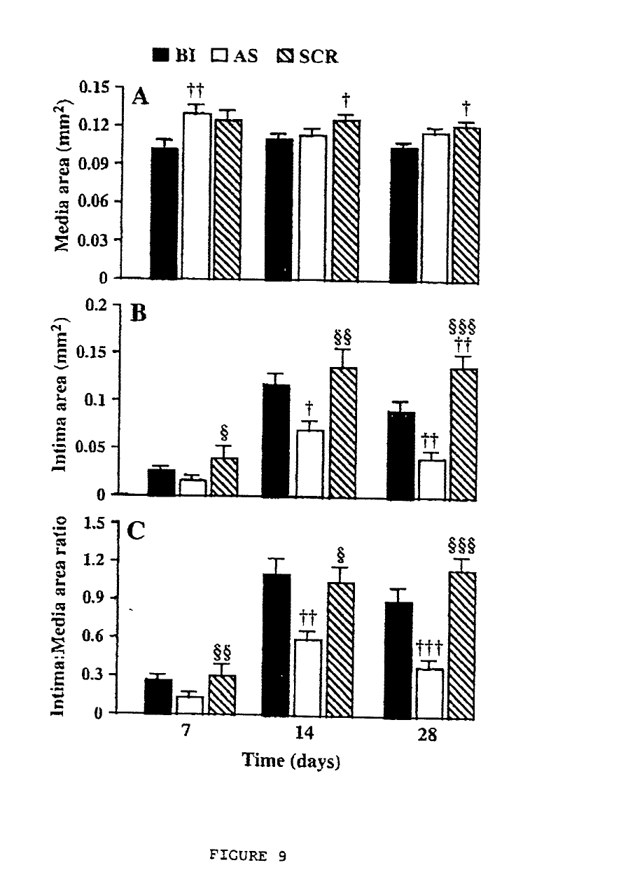

FIG. 9. Effect of AS-PDGFR-β on intimal hyperplasia. Medial area of vessels treated with AS-PDGFR-β or SCR oligomers was increased slightly compared with an injured, untreated artery (BI) (A). AS-PDGFR-β reduced neointimal area at 14 and 28 days after injury (B). I:M area ratio was reduced by application of AS-PDGFR-0 at days 14 and 28 after injury (C). n-5 to 25 animals per treatment. Symbols as in FIG. 8.

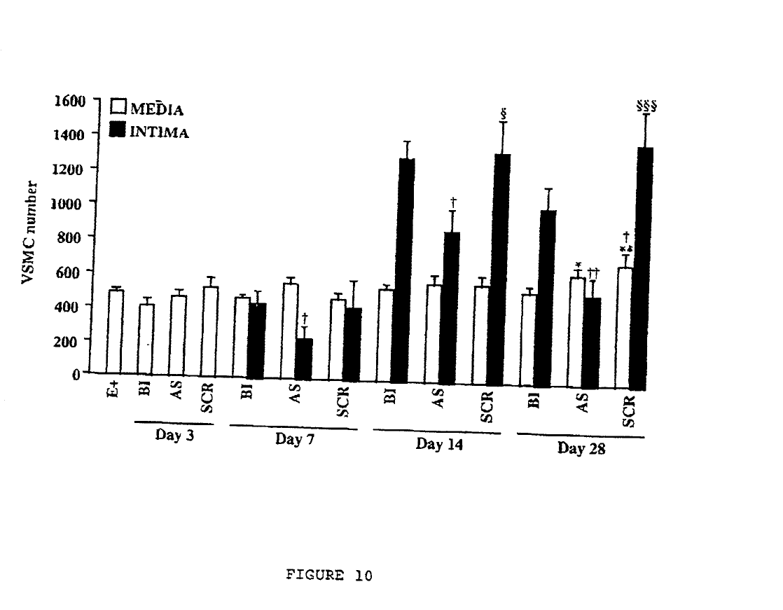

FIG. 10. Quantification of VSMC number in media and neointima of injured carotid arteries. AS-PDGFR-β reduced number of VSMCs in intima compared with BI group. n=4 to 16 animals per treatment. Symbols as in FIG. 8.

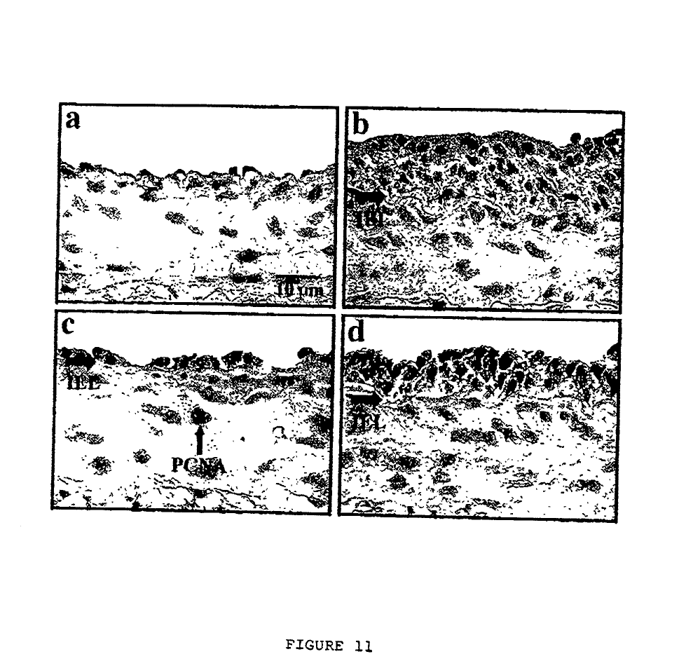

FIG. 11. PCNA protein expression 7 days after injury. Positive PCNA expression was detected by immunohistochemistry (cells stained in brown; vertical arrow). Baseline PCNA expression in native arteries was almost nil (a); it was overexpressed at day 7 in intima and media of injured arteries (b); AS-PDGFR-β (c) and SCR treatment (d) did not prevent PCNA protein overexpression. Internal elastic lamina is indicated (IEL; horizontal arrow).

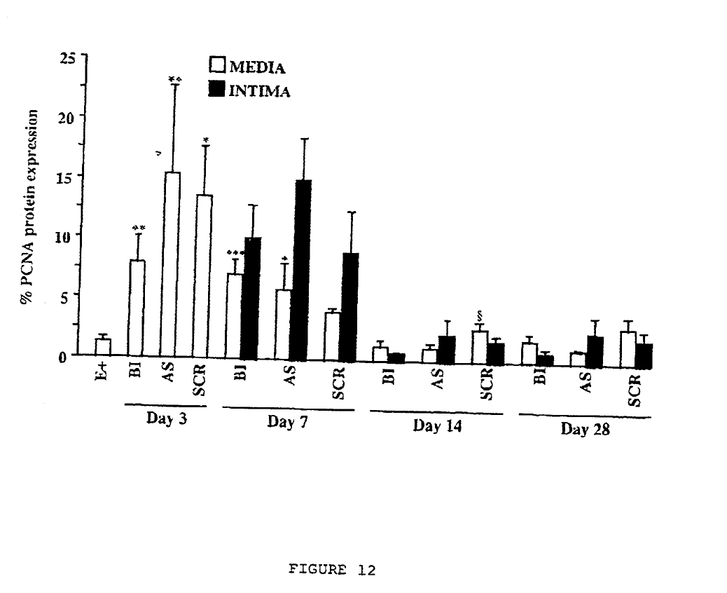

FIG. 12. Quantification of VSMCs in proliferative state. Base-line expression of PCNA protein in media of uninjured carotid arteries (E+) was 1.15%. Vascular injury induced PCNA overexpression in media at days 3 and 7 and in intima at day 7; values returned to baseline levels by day 14. Treatment with either AS-PDGFR-β or SCR oligomer did not prevent PCNA protein overexpression. n4 to 14 animals per treatment. Symbols as in FIG. 8.

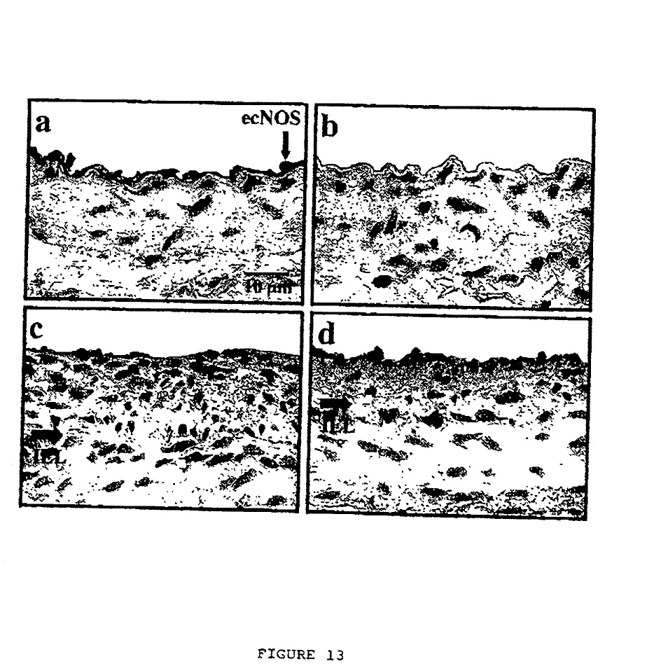

FIG. 13. ecNOS detection on injured carotid arteries. Positive ecNOS expression was detected by immunohistochemistry (cells stained brown; vertical arrow). Baseline ecNOS expression in native arteries was present on each endothelial cell (a), absent immediately after a vascular injury (b), and partial 28 days after injury (c). In AS-PDGFR-β-treated group, extent of reendothelialization was improved (d). Internal elastic lamina is indicated (IEL; horizontal arrow).

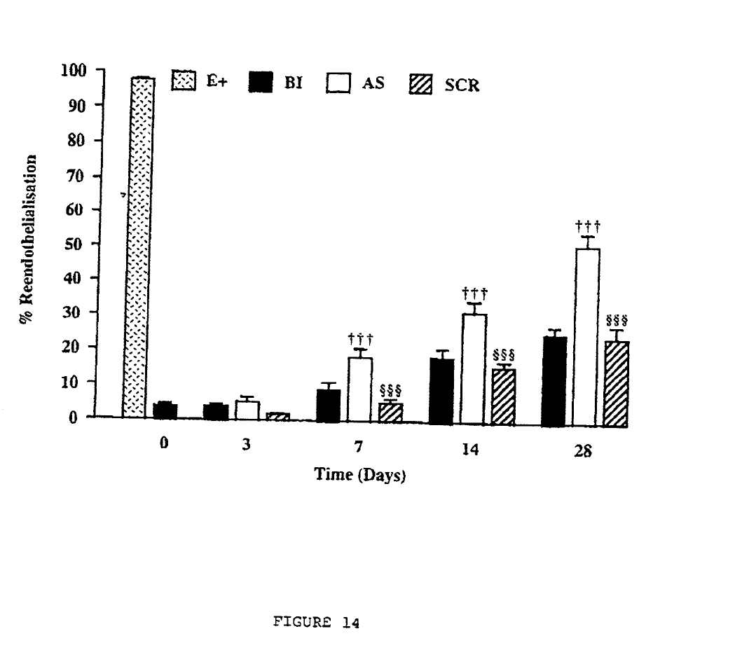

FIG. 14. Vascular reendothelialization of injured carotid arteries. Expression of ecNOS in uninjured carotid arteries (E+) covered 96.6% of luminal circumference of artery. Immediately after BI, endothelium covered 2.6% of vascular lumen. Treatment with AS-PDGFR-β improved reendothelialization process at each time point compared with BI group, whereas SCR oligomer had no beneficial effect. n=4 to 20 animals per treatment. Symbols as in FIG. 8.

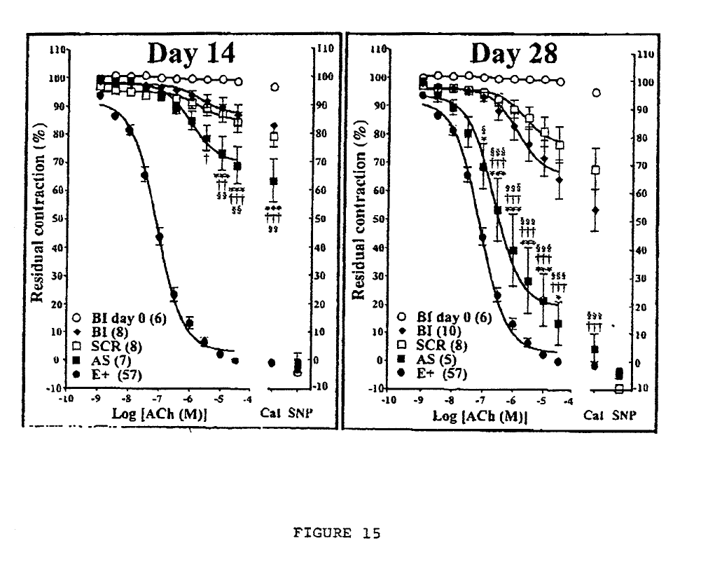

FIG. 15. Restoration of vascular reactivity. Results are expressed as percentage of residual contraction to PE. Normal vessels (E+) relax completely to ACh, whereas freshly denuded carotids (BI day 0) did not show any endothelium-dependent relaxation. At day 14, BI untreated and SCR-treated vessels had 15% relaxation to ACh, whereas treatment with AS-PDGFR-β doubled vasorelaxation to ACh. At day 28, BI, SCR, and AS groups relaxed by 24%, 36%, and 87%, respectively, under ACh 10-5 mol/L treatment. At day 28, ACh EC50 was 50 1.34×10-6 mol/L for BI vessels, 2.23×10-6 mol/L for SCR-treated, and 2.47×10-7 mol/L for AS-PDGFR-β-treated vessels. n=5 to 10 animals per treatment and n=57 for native arteries. Cal indicates calcium ionophore. Symbols as in FIG. 8.

### EXAMPLES

### Example 1

### PDGFR-β Expression Inhibition Directs Suppression of Intimal Thickening

Induction of Intimal Hyperplasia:

Balloon denudation of common carotid arterial endothelium was performed in male Sprague-Dawley (350-425 g) (Charles River Breeding Laboratories, Kingston, Mass.). The rats were anesthetized with intraperitoneal injections of ketamine HCl 75 mg/kg (Ketaset, Aveco Co, Fort Dodge, IA) and xylazine HCl 5 mg/kg (Xyla-ject, Phoenix Pharma., St. Joseph, Mo.) Following exposure of the left common external carotid artery, a 2 French Fogarty balloon catheter (American Edwards Laboratories, Santa Ana, Calif.) was inserted through an arteriotomy into the common carotid artery to the aortic arch, insuflated sufficiently with air to produce slight resistance and withdrawn three times. Upon removal of the catheter, the external carotid artery was ligated, the wounds were closed, and the animals were returned to their cages. Animals were sacrificed at different periods of time (7, 14, and 28 days) after injury with an overdose of ketamine and xylazine, exsanguinated and perfused with 50 ml of Ringer's lactate solution. The treated segment of the common carotid artery was removed, cut in 2 equal segments and fixed in 5% formalin solution. The segments were embedded in paraffin and eight sections of 6 μm were obtained by microtome along the length of the specimen. Sections were stained with Hematoxylin-Eosin and the areas of the intima, media and adventitia, the intima:media area ratio and the percent of luminal occlusion were calculated for each arterial segment using computerized digital planimetry with a dedicated video microscope and customized software. The nature of specimen treatment was kept from investigators until after completion of the data analysis.

Antisense Oligonucleotides Therapy:

To study the possible contribution of PDGFR-β subunit in neointima formation antisense oligonucleotide sequences to the receptor subunit were applied directly to balloon catheter denuded carotid arteries. We employed two different antisense oligonucleotide phosphorothioate backbone sequences to the murine PDGFR-β mRNA subunit (antisense 1 [AS1-PDGFR-β:TAT CAC TCC TGG MG CCC]; SEQ ID No: 1 nucleotides 4 to 21; and antisense 2 [AS2-PDGFR-β: TCT GAG CAC TM AGC TGG]; SEQ ID No. 2 nucleotides 22 to 39). Neither sequence contained more than two consecutive guanosines. Two scrambled phosphorothioate sequences (scramble 1 [SCR1 GTG ATA GTA TGC CGA GCA]; SEQ ID No: 3 and scramble 2 [SCR2 CGT TAC GTA AGC CTA GGA]; SEQ ID No: 4) were used as controls. All sequences were synthesized at the Massachusetts Institute of Technology Biopolymers Laboratory. The oligonucleotides were deprotected, dried down, resuspended in Tris-EDTA (10 mmol Tris pH 7.4 and 1 mmol EDTA pH 8.0), and quantified by spectrophotometry. To sustain the release and insure the local administration of the oligonucleotide sequences directly to the injured arteries the oligomers were embedded within ethylenevinyl acetate copolymer (EVAc; DuPont Co., Wilmington, Del.) matrix release devices as previously described (17, 24-26). After the endothelial denudation of the left common carotid artery, the EVAc devices containing 400 μg of the scrambled or antisense PDGFR-β oligomers were placed adjacent to the injured carotid arteries. In 14 days approximately 65% of the compound was released with a zero-order kinetics, and it has been estimated that approximately 1% of the released oligomer would be delivered to the blood vessel wall from these types of devices (17, 25).

Immunohistochemistry of PDGFR-αand -β Subunit Expression:

Expression of PDGFR-α and -β subunits was determined immunohistochemically. Arterial sections were deparaffinized in xylene and ethanol baths, endogenous peroxidase activity was quenched in a solution of methanol (200 ml) plus hydrogen peroxide (3%, 50 ml), and nonspecific binding antibody binding prevented by preincubating the tissues with serum (1:10) from species other than those used to raise the primary antibody. Arterial sections were then exposed to the primary antibody, PDGFR-α IgG (Santa Cruz Biotech., Santa Cruz, Calif.) or rabbit polyclonal anti-human PDGFR-β IgG (UBI, Lake Placid, N.Y.) diluted (1:100, 1:200, 1:500, 1:1000), or rinsed with PBS, and incubated with a biotinylated goat anti-rabbit IgG (1:400) (Dako, Carpinteria, Calif.). A Dot-blot and Western blot analysis were performed to confirm the cross reactivity of both rabbit antibodies to rat proteins. Peroxidase labelling was achieved with an incubation using avidin/peroxidase complex (Vector Labs Inc., Burlingame, Calif.), and antibody visualization established after a 5 min exposure to 0.05% 3,3′-diaminobenzidine (Sigma Chem, St Louis, Mo.) in 0.05 M Tris-HCl at pH 7.6 with 0.003% hydrogen peroxide. The arteries were counterstained by a rapid immersion (10 seconds) in Gill's hematoxylin #3 solution, and rinsed in tap and distilled water.

Cell Culture:

Vascular smooth muscle cells (vSMC) of rat thoracic aorta were isolated by the explant technique (27). The cells were seeded in culture dishes (35 mm), grown to confluence in Dulbecco's modified Eagle medium (DMEM) supplemented with 10% fetal bovine serum (complement-heat inactivated), penicillin (50 U/ml) and streptomycin (50 mg/ml), and used between the 6th and 10th passage. At confluence, the medium was replaced with DMEM, 0.1% FBS and antibiotics two groups of cells were treated either with AS1-PDGFR-β or SCR1-PDGFR-β (direct application not embedded into EVAc matrices) at 0, 24 and 48 hrs, whereas a third group was untreated and served as control. PDGF-BB (10 ng/ml) was added and total proteins from the cells were collected 0, 1, 3, 6, 12, 24 and 48 hrs later.

Western Blot Analysis of PDGFR-α and -β Protein Subunit:

Total proteins were prepared by washing the cells with ice cold PBS, and the addition of 100 μl of Laemmli buffer containing EDTA 1 mM, phenylmethylsulfonyl fluoride 1 mM, leupeptin 10 μg/ml and NaVO₃ 1 mM. The extracted cell proteins were boiled for 5 min, and a 30 μl aliquot (˜30 μg protein) of each sample was separated by 7.5% sodium dodecyl sulfate polyacrylamide gel electrophoresis (SDS-PAGE) under reducing conditions (Minigel Apparatus, Bio-Rad) and transblotted onto 0.45 μm polyvinylidene difluoride membranes (PVDF, Millipore). The membranes were blocked in TBS-5% Blotto (Tris-HCl 10 mM, NaCl 150 mM pH 7.85: 5% non fat dry milk Bio-Rad) for 1 hr at room temperature with gentle agitation. Membranes were washed with TBS and TTBS (TBS; 0.05% Tween 20 Bio-Rad), and incubated with rabbit polyclonal anti-human PDGFR-β IgG antibodies (dilution 1:200 in TTBS) for 2 hrs at room temperature. The membranes were washed with TTBS and incubated with alkaline-phosphatase goat anti-rabbit IgG (1:100) for 2 hrs at room temperature. Membranes were washed with TTBS and TBS and alkaline phosphatase bound to secondary antibodies was revealed by chemiluminescence (Bio-Rad Kit). Prestained molecular weight marker proteins (Bio-Rad) were used as standards for SDS-PAGE. To probe the immunoblots with second antiserum, the PVDF membranes were stripped by incubation In 62.5 mM Tris-HCl, pH 6.7, 2% SDS, and 100 mM 2-mercaptoethanol for 30 min at 50° C., gentle agitation. The blots were then washed twice with TBS, then washed at least 5 times to remove traces of 2-mercaptoethanol. Then, the blots were incubated with polyclonal anti-human PDGFR-α antibodies (dilution 1:200 in TTBS) and processed as described above.

Statistical Analysis:

Data are mean ±SEM. Statistical comparisons were determined by variance analysis followed by an unpaired Student's t-test with Bonferroni's correction for multiple comparisons. Data were considered to be significantly different if P<0.05 was observed.

Results

Neointimal Hyperplasia:

Effects of PDGFR-β mRNA antisense oligonucleotides: Neointimal formation determined 14 days after balloon deendothelialization of rat common carotid arteries served as controls for all subsequent experiments. At this time an intima:media area ratio of 1.37±0.15 was observed (FIG. 1). Antisense sequences directed against the PDGFR-β subunit mRNA were used to reduce receptor subunit expression. The sustained release of antisense oligonucleotide sequences AS1-PDGFR-β or AS2-PDGFR-β from EVAc matrices placed adjacent to the injured artery reduced the intima:media area ratio to 0.27±0.09 and 0.55±0.11, but neither scrambled oligonucleotide sequence significantly affected neointimal thickening (SCR1 1.5±0.12 and SCR2 1.66±0.13, FIG. 1). Medial areas were no different in any treated or control groups (data not shown). Protein expression of PDGFR-α and -β subunit: In the absence of vascular injury basal expression of PDGFR-β subunit was observed on medial vSMC. 26.5±2.5% of these cells were immunohistochemically identified with an antibody that specifically recognizes the PDGFR-β protein (FIGS. 2, 3A). 14 days after a denuding injury, PDGFR-β protein doubled on medial vSMC (51.2±5%, p<0.001) and became evident on 74.5±2.5% of the intimal cells (FIGS. 2, 3B). The perivascular sustained release of both antisense sequences significantly reduced PDGFR-β subunit expression in both vascular compartments, yet the sequence closer to the 5′ mRNA end, AS1-PDGFR-β, was more potent at reducing receptor subunit and neointima formation. Two weeks after the treatment of vascular injured carotid arteries with AS1-PDGFR-β, only 4.4±1.8% of medial cells and 2.8±1.6% of intimal cells retained PDGFR-P subunit expression (p<0.001 compared with controls (BI), FIGS. 2, 3C). The AS2-PDGFR-β oligonucleotide reduced these values to 15.9±5.2% and 19.1±5.2% respectively (p<0.001 compared with controls (BI), FIGS. 2, 3D). Scrambled oligonucleotide sequences had no effect on receptor subunit expression (data not shown). The suppression of neointima with application of antisense PDGFR-β oligomers followed inhibition of PDGFR-β subunit expression in an exponential fashion (Intima: Media area ratio=e^{(β/T)}); where β was the percent of all cells expressing the PDGFR-β subunit and T was defined as the exponential constant. intimal thickening correlated with medial PDGFR-β subunit expression with an exponential constant (T) of 17.64 (p>0.01; r=0.82, FIG. 4A), and with intimal receptor expression with an exponential constant (T) of 0.32 (p<0.001; r=0.96, FIG. 4B).

Specificity of the antisense oligonucleotide effect for PDGFR-β mRNA was demonstrated through similar immunohistochemical identification of PDGFR-α subunit protein expression. In absence of vascular injury PDGFR-α subunit expression was observed on 32.8±4.6% of medial vSMC (FIGS. 5, 6A). Fourteen days after denuding injury PDGFR-α expression increased on medial vSMC (52.7±3.4%. p<0.001) and was noted on 57.3±4.2% of the intimal cells (FIGS. 5, 6B). Despite their effects on PDGFR-β subunit expression the sustained perivascular release of either antisense sequences for 14 days after a vascular injury did not affect the PDGFR-α subunit expression. PDGFR-α protein expression in the media and intima of rat carotid treated with AS1-PDGFR-β was 58.5±3.2% and 61.5±2.8% respectively (FIGS. 5, 6C), and 59.4±3.5% and 62.9±3.8% respectively for animals treated with AS2-PDGFR-β (FIGS. 5, 6D). Treatment with scrambled oligonucleotide sequences did not alter the expression of PDGFR-α as compared to control animals (BI) (data not shown).

Protein Expression of PDGFR-β Subunit on Cultured Smooth Muscle Cells:

vSMC were grown to confluency on 35 mm Petri dishes, then kept quiescent in DMEM with 0.1% FBS, AS1-PDGFR-β (20 μM) or SCR1-PDGFR-β oligonucleotide (20 μM) were added at 0, 24 and 48 hrs, a third group of cells was untreated with oligonucleotide and served as control. Two days after the first oligonucleotide application, PDGF-BB (10 ng/ml) was added in each group. At 0, 1, 3, 6, 12, 24 and 48 hrs after the addition of PDGF-BB, the cells were washed with cold PBS, Laemmli buffer (100 μl) was added, total proteins collected, quantified by bioassay, and the expression of PDGFR-β at each time point was determined by Western blot electrophoresis and quantified by image densitometry. Significant basal PDGFR-β protein expression was noted in vSMC (FIG. 7). These values decreased by 53% one hour after stimulation with PDGF-BB, and by an additional 32% 11 hrs after that, to be reexpressed near basal levels 48 hours after initial stimulation. AS1-PDGFR-β suppressed protein expression by over 75% at baseline, and for the duration of the experiment (FIG. 7). These effects were specific for the PDGFR-β target gene as PDGFR-α protein expression was unaffected by the antisense PDGFR-β oligonucleotide sequence. The SCR1-PDGFR-β oligonucleotide sequence had no affect on the normal pattern of PDGFR-β protein expression seen in control vSMC at baseline and following stimulation with PDGF-BB (data not shown).

Discussion

In previous reports we and other investigators showed that antisense oligonucleotide sequences complementary to DNA binding proteins and cell-cycle regulators genes such as c-myb, c-myc, cdc2, cdk2, nonmuscle myosin and PCNA inhibited target protein expression, suppressed vSMC proliferation in vitro and in vivo and inhibited neointimal formation in injured arteries of different animal species (10-17, 28-31). To date, targeted genes were principally those involved in cell cycle progression. However, these genes are not unique to vSMC, but are also expressed in other cell types and their use might induce side effects in tissues with high rates of proliferation. Growth factors play a central role in all phases of the vascular response to injury, and yet no studies have yet to be reported on the consequences of antisense sequences directed against growth factor and/or their receptors. PDGF, for example, is critical to vSMC migration and intimal thickening (1, 7, 32) in a manner fairly selective for vSMC (7, 8, 32) and as a result became the focus of the present manuscript.

We employed two antisense oligonucleotide sequences selective for either positions 421 or 22-39 of the PDGFR-β mRNA subunit. As PDGFR-β subunit expression is reexpressed after initial down-regulation following PDGF-BB stimulation in vitro (FIG. 7) and is manifest over the full 2 week period after in vivo injury (FIGS. 2, 3), the oligonucleotides were embedded in EVAc matrices to provide a sustained release during the entire experimental procedure. Previous studies demonstrated the need to match the kinetics of oligonucleotide release to the kinetics of antisense target gene expression. When gene expression is prolonged, as it is for c-myc, a more sustained oligonucleotide release device was required to demonstrate biologic effect (17). Sustained release of the two antisense oligonucleotide sequences complementary to PDGFR-β mRNA reduced arterial intimal thickening by 80 and 60% respectively. In normal rat carotid arteries approximately 25% of the medial vSMC stained positively for PDGFR-β subunit protein. Two weeks after vascular injury this expression more than doubled in medial vSMC, and close to 75% of the cells forming the neointima stained positively as well. Interestingly, while both antisense sequences reduced PDGFR-β subunit protein expression below the basal level (25%) observed in the media of uninjured rat carotid arteries, the oligonucleoticde sequence closer to the 5′-mRNA region was almost four times more potent at inhibiting PDGFR-β expression in medial and intimal vSMC. The variable response to these two sequences enabled delineation of a correlation between PDGFR-β levels and neointimal potential. In arteries where PDGFR-β subunit expression was reduced below basal levels, i.e., in fewer than ˜25-30% of all cells, only minimal intimal thickening was observed. When PDGFR-β subunit expression exceeded basal levels, intimal proliferation rose exponentially (FIG. 4).

Though the first antisense sequence (AS1) almost completely reduced PDGFR-β subunit protein expression by day 14, it did not completely abolish intimal hyperplasia This observation raises the possibility that while PDGFR-BB stimulation may contribute up to 80% of the neointima formation, the secretion of other growth factors or peptides might contribute to the residual fraction (33 36) Alternatively, the lack of complete inhibition of neointima may stem from the inability of the sustained antisense delivery system to fully suppress the immediate-early PDGF effect. The EVAc matrices allow the release of their embedded contents over the entire course of the experiment, not as a large bolus at the time of injury. Upon vascular injury the almost immediate platelet adhesion to subendothelial connective tissue induces the release of platelet PDGF-BB which stimulates its PDGFR-BB, and the interval of time between balloon denudation and oligonucleotide release upon application may well have allowed sufficient growth factor-receptor interaction to activate the intracellular events that lead to neointima formation. Indeed, our In vitro study revealed first, a complex pattern of PDGFR-β protein expression in response to stimulation with PDGF-BB, with initial suppression of heightened basal levels that returned within 48 hrs, and second, that pretreatment with AS1-PDGFR-β oligonucleotide reduced receptor subunit expression at baseline by 4 fold, and upon stimulation with PDGF-BB for the duration of the experiment (FIG. 7). The administration of antisense PDGFR-β oligomers days before the surgical procedure might reduce the basal expression of PDGFR-β subunit sufficiently to prevent its interaction with PDGF-BB or prevent the biological activity induction related to their interaction after the injury. Such studies could also allow one to determine the impact of these early interactions to residual intimal thickening.

The use of antisense technology is beset by questions of specificity (37-39). Recent reports have raised concern that the antiproliferative activity of antisense oligonucleotides to c-myb and c-myc, for example, was arose from aptameric rather than a hybridization-dependent antisense mechanism (37, 38). It was hypothesized that oligonucleotides with four sequential guanosines might bind to serum proteins including growth factors such as bFGF, aFGF, PDGF and VEGF, reducing the interaction of these growth factors with their receptors, and the intracellular signal transduction leading to gene protein expression (such as c-myc and c-myb) involved in cell cycle progression (39). Nonetheless, other studies have shown specific in vivo and/or in vitro effects with antisense oligonucleotides lacking multiple sequential guanosines to these and other genes involved in cell cycle progression such as cdc2, cdk2, nonmuscle myosin and PCNA (11-14, 28). Neither antisense sequence used in the studies we now report possessed more than two contiguous guanosines. To more definitely address this issue however, we examined the effects of the sequences on the a subunit. As antisense sequence can discriminate between oligonucleotide sequences that differ by one or two bases (15, 40, 41) we compared effects of AS1 on the PDGFR-α and -β subunits. Quantitative analysis of protein expression on vSMC in culture confirmed immunohistochemical identification of antigenicity in vivo. The antisense sequences directed against the -β subunit inhibited only this targeted protein subunit without affecting the PDGFR-α protein subunit expression (FIGS. 5-7). Scrambled oligonucleotide sequences also failed to reduce neointima formation, or PDGFR-β subunit protein expression in vitro or in vivo.

It is interesting to note that the antisense sequence closer to the 5′ end of PDGFR-β mRNA was more potent at inhibiting intimal thickening and PDGFR-β protein expression than the AS2 sequence. This is in accordance with previous reports which have shown that the biologic effects of antisense oligomers are dictated in part by the location of the sense target sequence. Antisense oligonucleotides directed at or near the 5′ translation initiation site were most effective at inhibiting gene expression, and in some cases a shift of few base pairs in the targeted sequence was sufficient to induce drastic variation in target gene inhibition (42-45). This discrepant effects between similar sequences remains enigmatic. Possible explanations could be that the secondary structure of the mRNA close to the initiation codon might offer a more favourable hybridization site for the antisense sequence. Downstream regions of the mRNA might fold and reduce the hybridizing access for the antisense sequences. Alternatively, antisense sequences complementary to or near the 5′ mRNA region may be more potent at preventing mRNA translation (46-48). These and other issues will require further study before antisense technology can reach its full potential.

Conclusion

We observed that the sustained perivascular application of antisense oligonucleotide sequences complementary to PDGFR-β mRNA not only prevented overexpression of PDGFR-β protein in healing medial and intimal vSMC but did so in a manner commensurate with effects on intimal thickening. Almost complete abolition of PDGFR-β protein expression was achieved with the antisense sequence closer to the 5′ PDGFR-P mRNA. The antisense PDGFR-β effect was specific. The oligomers employed did not bear 4 contiguous guanosines eliminating concern for non-specific, aptameric binding, and only the antisense sequences suppressed protein expression, and only of the target PDGFR-β, and not the PDGFR-α protein subunit.

As PDGFR-β expression is specific to mesenchymal cells such as vSMC and fibroblasts, the regulation of this cell membrane receptor might provide an important advantage over the inhibition of cell cycle proliferative proteins which are expressed ubiquitously. Regulation of PDGFR-β could contribute to the prevention of intimal thickening without affecting the proliferation of unrelated but critical cells. Further investigations are needed to determine whether and how the neointima will respond with release of PDGFR-β protein expression inhibition after the removal or the degradation of the antisense oligomers. Finally, our results demonstrate again the value of antisense technology in helping elucidate the mechanisms involved in vascular healing, and as a possible approach to the prevention and progression of the accelerated arteriopathies that follow vascular intervention.

It is readily apparent from the foregoing that antisense oligonucleotides to PDGFR-β mRNA successfully prevented restenosis. Other antisense oligonucleotides may be designed from the sequences of the receptor PDGFR-β subunit and used with success. The antisense may be adjuncted with any other antisense oligonucleotides which also show inhibition of intimal thickening. Examples thereof are those already described in WO 93/08845 and U.S. Pat. No. 5,593,974 which hybridize with c-myb (SEQ. ID. No. 5) NMMHC (SEQ. ID. No. 6) and/or PCNA (SEQ. ID No. 7) mRNAs.

### Example 2

### Bolus Endovascular PDGFR-β Antisense Treatment Suppressed Intimal Hyperplasia While Favorising Reendethelialization

Induction of Intimal Hyperplasia

BI of common carotid arterial endothelium was performed in male Sprague-Dawley rats (325 to 400 g) as described above. Animals were euthanized at different periods of time (0, 3, 7, 14, and 28 days) after injury with an overdose of Ketamine and xylazine, exsanguinated, and perfused with 100 mL of Ringers lactate solution by the left ventricle. The left (treated) and right (untreated)segments of the common carotid arteries were removed and fixed in 10% formalin PBS. The segments were embedded in paraffin, cut into 6-μm longitudinal sections, and stained with Masson's trichrome solution. The areas of the intima and media and the intima-to-media (I:M) area ratio were calculated by computerized digital planimetry.

Antisense Oligonucleotide Therapy

We used an AS oligonucleotide phosphorothioate backbone sequence to the murine PDGFR-β mRNA subunit (AS-PDGFR-β: TATCACTCCTGGAAGCCC; SEQ ID NO.: 1). A scrambled (SCR) sequence (SCR-PDGFR-β: GTGATAGTATGCCGAGCA; SEQ ID NO.: 3) was used as control. After BI of the left common carotid artery, we introduced a 22-gauge infusion cannula into the external carotid arteriotomy and administered 0.2 mL of 0.9% NaCl solution to flush the residual blood-borne elements. The AS or SCR oligonucleotide solution (200 μg/25 μL of PBS 0.01 mol/L) was infused into the temporarily isolated segment of the left common carotid artery for a 30-minute period. Then the arteriotomy was ligated, the left common carotid artery was released, the wounds were closed, and the animals were returned to their cages. The protocol was performed in accordance with the Canadian Council on Animal Care guidelines.

Evaluation of Vascular Reactivity

Carotid arteries were harvested at death and placed in Krebs-Ringer solution. Rings of 4 to 5 mm from the media] portion of the left (treated) and right (untreated) carotids were mounted with 2 triangle 5-0 stainless steel wires. The adjacent segments (distal and proximal) were fixed in formalin for analysis. Experiments were performed in organ chambers filled with 25 mL of Krebs-Ringer solution and indomethacin 0.01 mmol/L and gassed with 95% 0215% CO₂ at 37° C. Vessels were passively stretched (≈1.5 g) while the contraction generated by a depolarizing solution containing physiological KCl (20 mmol/L) was assessed The organ chamber was rinsed with fresh Krebs-Ringer solution and equilibrated for 45 minutes. Phenylephrine (PE; 10⁻⁶ mol/L) was used to achieve a submaximal contraction. An endothelium-dependent vasorelaxation was induced by the addition of cumulative acetylcholine (ACh) concentrations (10⁻⁹ to 3.17×10⁻⁵ mol/L). Calcium ionophore A23187 (2.5×10⁻⁷ mol/L) was added to obtain the maximal endothelium-dependent vasoretaxation. Sodium nitroprusside (10⁻⁵ mol/L) was added to mediate a direct VSMC relaxation.

Immunohistochemistry of pdgfr-β, pcna, and ecnos Expression

The immunohistochemistry procedures on arterial sections were performed as described above. The primary antibodies used were rabbit polyclonal anti-human PDGFR-β IgG (UBI), monoclonal anti-human proliferative cell nuclear antigen (PCNA) IgG (Zymed Laboratories Inc), and monoclonal anti-human endothelial cell constitutive nitric oxide synthase (ecNOS) IgG (Transduction Laboratories)].

Statistical Analysis

Data are mean±SEM. Statistical comparisons were determined by ANOVA followed by an unpaired Student's t test with Bonferroni's correction for multiple comparisons. Data were considered significantly different if a value of P<0.05 was observed. Relaxation is expressed as a percentage of preconstricting tone. EC₅₀ (concentration of ACh producing a half-maximal relaxation) has been calculated for each segment with the Statview program.

Results

Expression of PDGFR-β Protein Subunit

In native arteries, basal expression of the PDGFR-β subunit was observed immunohistochemically on 1.4±0.4% of medial VSMCs (FIG. 8). PDGFR-β protein increased 8.7-fold in medial VSMCs (P<0.001) by day 3 after injury, reached a plateau at day 7 (12.6-fold increase, P<0.001), and returned to basal levels by day 14 (FIG. 8). The presence of intimal VSMCs was observed by day 7 after injury, with 18.3±3.7% of intimal VSMCs staining positively for PDGFR-0 protein. By day 14, the PDGFR-β protein expression in intimal VSMCs returned to basal level (FIG. 8).

Treatment with AS-PDGFR-β prevented PDGFR-β protein overexpression in medial VSMCs at days 3 and 7 by 90% and 93%, respectively (P<0.001). Similarly, PDGFR-β protein level was reduced by 60% (P<0.05) in intimal VSMCs at day 7 and was at the basal level observed in native medial VSMCs at day 14 (FIG. 8). Three days after injury, treatment with an SCR oligomer reduced the PDGFR-β protein expression on medial VSMCs by 42% (P<0.05). This reduction, however, was significantly less (P<0 05) than the reduction mediated by the AS-PDGFR-0 (90%) (FIG. 8). At day 7, SCR treatment did not reduce PDGFR-0 protein expression in medial or intimal VSMCs, and by day 14 the PDGFR-β protein expression returned to basal levels (FIG. 8).

Neointimal Hyperplasia

The intimal and medial areas (mm²) and the I:M area ratio were determined after a vascular injury. The medial areas in BI rat carotid arteries at days 7, 14, and 28 after injury were 0.101±0.007, 0.109±0.005, and 0.105±0.004 mm², respectively (FIG. 9A) and fluctuated by <14% compared with the medial area of native carotid arteries (data not shown). Treatment of the BI carotid arteries with AS-PDGFR-β increased the medial area by 33%, 3%, and 13% at days 7, 14, and 28, respectively (P<0.01 at day 7 and P=NS at days 14 and 28). SCR treatment increased the medial area by 23%, 14%, and 16.5% (P=NS at day 7 and P<0.05 at days 14 and 28) (FIG. 9A). Intimal hyperplasia developed during the first 7 days and was maximal within 14 days. The intimal areas in BI groups at days 7, 14, and 28 were 0.025±0.005, 0.116±0.012, and 0.091±0.011 mm² (FIG. 9B). An AS-PDGFR-β treatment reduced the intimal hyperplasia by 37%, 40%, and 56% (P=0.07 [NS], P<0.05, and P<0.01) at days 7, 14, and 28, respectively, whereas the SCR treatment did not reduce the intimal hyperplasia (FIG. 9B). The I:M area ratios in BI carotids were 0.256±0.047, 1.102±0.126, and 0.899±0.099, respectively (FIG. 9C). An AS-PDGFR-β treatment reduced these ratios by 50%, 47%, and 58% (P=0.08 [NS], P<0.01. P<0.001), respectively, whereas the SCR treatment did not significantly alter the I:M area ratios compared with BI groups (FIG. 9C).

SCM Count

The induction of a carotid BI did not affect the medial VSMC count throughout the first 14 days compared with native vessels (467±38 cells) (FIG. 10). At day 28 after injury, however, all groups demonstrated an increased number of medial VSMCs compared with native media. The VSMC count increased by 11% (P=NS) in the untreated BI group, by 32% (P<0.05) in the AS-PDGFR-β-treated group, and by 47% (P<0.01) in the SCR-treated group. The difference between the AS-PDGFR-β and the BI groups was not significant (FIG. 10). At days 7, 14, and 28, the number of intimal VSMCs in BI arteries was 422±67, 1285±100, and 1004±126, respectively. AS-PDGFR-0 reduced the number of intimal VSMCs at days 7, 14, and 28 by 47%, 33%, and 50% (P<0.05, P<0.05, P<0.01), respectively, compared with the BI group. The SCR oligomer did not reduce the intimal VSMC count at any time point (FIG. 10).

SMC Density

The medial density of VSMCs in native carotid arteries was 4253±160 VSMCS/mm². The fluctuation density of medial VSMCs at days 3, 7, 14, and 28 after injury in BI or AS-PDGFR-β—or SCR-treated groups was always <20% compared with the VSMC density observed in native medial VSMCS. The variation of medial VSMC density between the BI group and the groups treated either with AS-PDGFR-β or SCR oligomer was also <20% (data not shown). The intimal VSMC densities in the B1 group at days 7, 14, and 28 after injury were 14 762±1143, 11 466±496, and 11 939±681 VSMCS/mm². The AS-PDGFR-β significantly reduced the intimal VSMC density by 29% only at day 7 (data not shown).

SMC Proliferative Activity

In native carotid arteries, the percentage of proliferative medial VSMCs was 1.2±0.4% (FIGS. 11A and 12) At days 3 and 7 in the BI group, PCNA expression on medial VSMCs increased to 7.8±2.4% (P<0.01) and 6.8±1.3% (P<0.001) compared with native medial VSMCs and returned to the basal level of PCNA expression observed in uninjured medial VSMCs by day 14 (FIGS. 11B and 12). Intimal VSMC PCNA expression was quantified from days 7 to 28 after injury. In the BI group, the percentage of PCNA expression at day 7 was 9.8±2.4%, and it returned to near basal expression by day 14 (FIGS. 11B and 12). A treatment with AS-PDGFR-13 or SCR oligomer did not significantly reduce PCNA overexpression on medial and intimal VSMCs compared with the BI group at any time point (FIGS. 11C and 11D and 12).

Vascular Reendothelialization

To evaluate the extent of reendothelialization, immunohistochemical staining was performed to detect the expression of ecNOS. In native carotid arteries, ecNOS-positive cells covered 96.7±0.5% of the internal elastic lamina (FIGS. 13A and 14). Immediately after the passage (3 times) of an inflated balloon, the degree of endothelialization (day 0) was reduced to 2.7±0.3% (FIGS. 13B and 14). In the BI group, reendothelialization occurred but remained incomplete (FIGS. 13C and 14). Treatment with AS-PDGFR-β increased the extent of reendothelialization at each time point compared with the Bi group (FIGS. 13D and 14). The application of SCR oligomer did not favor reendothelialization (FIG. 14).

Ex vivo Carotid Vascular Reactivity

Segments of carotid arteries were precontracted to submaximal level with PE (10⁻⁶ mol/L). PE-induced contraction in endothelium-intact native arteries (E+; 0.68±0.04 g) was less than in freshly denuded arteries (day 0; 1.3 8±0.12 g). At 14 and 28 days after injury, PE-induced contraction varied between 0.97±0.11 and 1.28±0.10 g in BI or AS-PDGFR-β—and SCR-treated arteries (data not shown).

On PE-precontracted arteries, ACh induced a complete relaxation of endothelium-intact segments (E+; FIG. 15). The relaxant effect of ACh, which was absent in freshly denuded arteries (BI day 0) and maximal on days 14 and 28, produced only 13.4±3.7% (day 14) and 36.1±6.8% (day 28) of vasorelaxation (FIG. 15). AS-PDGFR-β but not SCR significantly improved (time-dependently) the efficacy of ACh-induced relaxation compared with the BI group (FIG. 9). After the addition of the highest concentration of ACh (3.17×10⁻⁵ mol/L), the calcium ionophore A23187 (10⁻⁷ mol/L) was added to obtain the maximal endothelium-dependent vasorelaxation. The addition of A23187 to injured carotid arteries either untreated (BI) or treated with the AS-PDGFR-β or SCR oligomers never induced >10% relaxation at 14 and 28 days after injury (FIG. 15). Sodium nitroprusside (10⁻⁵ mol/L), which induces a direct VSMC relaxation, produced 100% relaxation in all treated groups (FIG. 15).

Discussion

In the present study, we show that a local endovascular delivery of AS-PDGFR-β at the injured carotid artery site not only reduced the formation of intimal hyperplasia but also enhanced reendothelialization and almost completely restored the endothelium-dependent relaxing function. It is also very interesting to note that such treatment prevented, rather than simply delaying, the overexpression of PDGFR-β protein, which normally peaks 7 days after injury. Finally, we showed that the reduction of intimal hyperplasia mediated by AS-PDGFR-β treatment was not due to a reduction of medial and/or intimal VSMC proliferative activity but rather was attributable to the inhibition of medial VSMC migration into the intima.

After a BI, PDGFR-β protein expression increased in the media and the neointima. This was maximal at day 7 and returned to its baseline level at day 14. These results are in agreement with previous reports that have shown transient PDGFR-β protein overexpression in rat and human injured arteries.^{23,49} Bilder et al⁵⁰ reported that a selective PDGFR-β tyrosine kinase inhibitor given orally twice a day for 28 days decreased by 30% the I:M area ratio in injured porcine coronary arteries. Banai et al⁵¹ showed that a local intravascular delivery of a PDGF-receptor tyrosine kinase blocker reduced by 40% the I:M area ratio of BI porcine femoral arteries. Finally, Hart et al⁵² showed that repeated intravenous administration of mouse/human chimeric anti-PDGFR-β antibodies combined with a sustained heparin delivery decreased the I:M area ratio by 40% in BI baboon saphenous arteries. In our study, the single-bolus endovascular application of AS-PDGFR-β was sufficient to prevent the overexpression of PDGFR-β protein throughout the entire 28 days of our experiment, and this might explain why our treatment was more efficient (58%) in reducing the development of intimal hyperplasia than the above-mentioned studies. In Example 1, the sustained perivascular application of AS-PDGFR-β reduced the I:M area ratio by 60% to 80%. Our present results suggest that a sustained release of the AS-PDGFR-β is not necessary to achieve its optimal biological effect and reinforce the concept that the blockade of initial events after acute vascular injury might be sufficient to have prolonged benefits.^{17 52}

We calculated the number of medial and intimal VSMCs and their density per square millimeter (VSMCS/mm²), as well as the VSMC proliferative activity in the different groups studied. Although medial VSMC count was increased 28 days after injury in all 3 groups, medial VSMC density at each time point in BI and AS-PDGFR-β—or SCR oligomer-treated groups never fluctuated by >20% compared with VSMC density observed in the media of native carotid arteries. AS-PDGFR-β treatment reduced the number of intimal VSMCs at days 7, 14, and 28 by up to 50% compared with the BI group without altering intimal VSMC density at days 14 and 28. In addition, a treatment with either the AS-PDGFR-β or the SCR oligomer did not significantly reduce PCNA overexpression at any time point in medial and intimal VSMCs as observed in the BI group (FIGS. 11 and 12). These results demonstrate that the treatment of an injured rat carotid artery with AS-PDGFR-β did not alter the proliferative activity of the medial or intimal VSMCS. Thus, the reduction in intimal VSMC number and the I:M area ratio is attributed to the inhibition of medial VSMC migration into intima.

We observed that the passage of an inflated balloon in rat carotid arteries led to an almost complete denudation of the endothelium. In the untreated BI arteries, a progressive reendothelialization was achieved, but <25% of the luminal area was covered by day 28. The application of AS-PDGFR-β increased the extent of reendothelialization by 2-fold at each time point, such that nearly 50% of the neointima was covered by neoendothelial cells at 28 days. This result, combined with a 58% reduction of the I:M ratio observed in the same carotid arteries treated with AS-PDGFR-β, supports the hypothesis that the inhibition of VSMC migration from the injured media has the double beneficial effects of reducing intimal hyperplasia and improving the vascular healing process.

Finally, our results demonstrate that the contractile (PE) and relaxant (sodium nitroprusside) properties of VSMCs were unaltered by the different treatments. Most importantly, at 14 days and more convincingly at 28 days after injury, AS-PDGFR-β treatment significantly improved endothelium-dependent relaxation. The maximal relaxation produced by ACh more than doubled, and the estimated concentration of ACh needed to induce 50% of its maximal relaxation was reduced by 2- and 5-fold at 14 and 28 days, respectively, compared with injured untreated carotid arteries. Our results suggest that a 50% reendothelialization of injured rat carotid arteries might be sufficient to induce an almost complete endothelium-dependent vasorelaxation as observed in native arteries.

Conclusion

In conclusion, we have shown that the local endovascular delivery of a single bolus of AS-PDGFR-β at the injury site is sufficient to block the initial and delayed PDGFR-β protein overexpression, reduce the formation of intimal hyperplasia, and improve the degree of reendothelialization sufficiently to restore endothelium-dependent relaxant function to the injured carotid arteries. These data demonstrate the clinical potential of AS-PDGFR-β to prevent accelerated arteriopathies and promote vascular healing of injured areas.

### Example 3

### Antisense Molecules Directed Against Other Targets Reduce SMC Proliferation

It has been shown that, in vitro, antisense oligonucleotides to both c-myb (Seq. ID No. 5) and NMMHC (Seq. ID No. 6) caused substantial suppression of cellular proliferation while the sense oligonucleotides had no effect and were similar to the results obtained using just Tris-EDTA buffer as a control.

Antisense c-myb oligonucleotide:

Sequence ID No. 5

GTGTCGGGGTCTCCGGGC

Antisense NMMHC oligonucleotide:

Sequence ID No. 6

CATGTCCTCCACCTTGGA

The inhibitory action of antisense phosphorothiolate oligonucleotides directed against NMNHC or c-myb was concentration-dependent (antisense NMMHC: 32% vs 65% suppression at 2 μM and 25 μM, respectively; antisense c-myb: 33% vs 50% suppression at 2 μM and 25 μM respectively). Previous estimates of the relative abundance of these two messages indicated that c-myb mRNA occurs at extremely low concentrations in exponentially growing SMC (less than 0.01% of poly A+RNA), whereas NMMHC mRNA is present at significantly higher levels. The observed concentration dependence of the two antisense oligonucleotides with regard to growth inhibition was consistent with the relative abundance of the two mRNAs.

The antiproliferative effects of the antisense and sense phosphorothiolate oligonucleotides were also evaluated with the BC3H1 cell line as well as with primary rat and mouse aortic SMC. The data obtained showed that growth of the three cell types is greatly suppressed with phosphorothiolate antisense but not sense NNMHC or c-myb oligonucleotides. The admixture of antisense c-myb oligonucleotides for 4 hr produced an antiproliferative effect which is identical to that observed with continuous exposure for 72 hr (50% suppression of cell growth).

The treatment of SMC with antisense NMMHC oligonucleotides produced no growth inhibitory effect at either time point, whereas exposure to antisense c-myb oligonucleotides generated a 19% suppression of proliferation at 72 hr and a 40% suppression of proliferation at 120 hr.

### Example 4

### Release of Oligonucleotides from Polymeric Matrices

Release of Oligonucleotides from Pluronic™ Gel Matrix

Matrices made from a poly(ethylenoxide-propylene oxide) polymer containing c-myb and NMMHC antisense oligonucleoticles (described in Materials and Methods) were prepared in order to test the rate of release of the oligonucleotides from the matrices. The test samples were prepared by weighing 1.25 g of UV sterilized Pluronic™ 127 powder (BASF Corp., Parsippany, N.J.) in scintillation vials and adding 3.25 ml of sterile water. Solubilization was achieved by cooling on ice while shaking. To these solutions were added 500 μl of a sterile water solution containing the oligonucleotides (5.041 mg/500 μl). The final gels contained 25% (w/w) of the polymer and 1 mg/g oligonucleotides.

The release kinetics of the gels containing oligonucleotides were determined by placing the gels in PBS and measuring the absorption (OD) over time. The results for four test gels indicate that oligonucleotides are released from the gels in less than one hour.

Release of Oligonucleotides from EVAc Matrices

The release of oligonucleotides from ethylene vinyl acetate (EVAc) matrices was demonstrated.

Matrices were constructed and release was determined as described by Murray et al. (1983), In Vitro., 19: 743-748. Ethylene-vinyl acetate (EVAC) copolymer (ELVAX 40P, DuPont Chemicals, Wilmington, DB) was dissolved in dichloromethane to form a 10% weight by volume solution. Bovine serum albumin and the oligonucleotide were dissolved together at a ratio of 1000-2000 1 in deionized HO frozen with liquid N end then lyophilized to form a dry powder. The powder was pulverized to form a homogeneous distribution of particles less than 400 microns in diameter. A known quantity of the powder was combined with 4-10 ml of the 10% (w/v) EVAc copolymer solution in a 22 ml glass scintillation vial. The vial was vortexed for 10 seconds to form a homogeneous suspension of the drug particles in the polymer solution. This suspension was poured onto a glass mold which had been precooled on a slab of dry ice. After the mixture froze it was left in place for 10 minutes and then removed from the mold and placed into a −20° C. freezer for 2 days on a wire screen. The slab was dried for an additional 2 days at 23° C. under a 600 millitorr vacuum to remove residual dichloromethane. After the drying was complete 5 mm×0.8 mm circular slabs are excised with a #3 cork borer.

The results indicate that about 34% of the oligonucleotide was released within the first 48 hours.

### Example 5

### In vivo Application of Oligonucleotides to Inhibit c-myb and NMNHC in Rats

Animal Model.

Balloon stripping of the rat carotid artery is used as a model of restenosis in vivo. Rats were anesthetized with Nembutal (50 mg/kg). A left carotid dissection was carried out and a 2F Fogarty catheter was introduced through the arteriotomy incision in the internal carotid artery. The catheter was advanced to the aortic arch, the balloon was inflated and the catheter withdrawn back to the arteriotomy site. This was repeated two more times. Subsequently, the balloon being withdrawn, the internal carotid was tied off, hemostasis achieved, and the wound closed.

Oligonucleotide Delivery.

The oligonucleotides were applied with a hydrogel and with an implantable ethylene vinyl acetate (EVAc) matrix. A polyethylene oxide-polypropylene oxide polymer (Pluronic™ 127, BASF, Parsippany, N.J.) was used as a hydrogel. The Pluronic™ gel matrices were prepared as described in Example 3. Briefly, sterile solutions of Pluronic™ 127 were prepared by weighing 1.25 g of UV sterilized Pluronic powder into a scintillation trial and adding 3.25 ml of sterile water. Solubilization was achieved by cooling on ice while shaking, forming a solution containing 27.7% by weight of the polymer. To these solutions were added 500 μL of a sterile water solution of the antisense c-myb (See Example 3) oligonucleotides (5.041 mg/500 μL). The final gels were 25% w/w of Pluronic™ polymer and 1 mg/g oligonucleotide. Drug-free 25% (w/w) gels were prepared as controls. The EVAc matrices were prepared as described in Example 4, and contained 40 μg of oligonucleotide.

Immediately after balloon injury, 200 μl of Pluronic/oligonucleotide solution (which contained 200 μg of the oligonucleotide) was applied to the adventitial surface of the artery and gelling was allowed to occur. The antisense/EVAc matrix (which contained 40 μg of the oligonucleotide) and drug-free gels were applied in the same manner.

Quantification of Effect.

After 14 days, the animals were sacrificed and the carotid arteries were perfused under pressure (120 mmHg with Ringer s Lactate. Both carotid arteries were excised and fixed in 3% formalin. Thin sections were then prepared for light microscopy in a standard manner. The slide was visualized and digitized using a dedicated computer system and by a hand held plenymeter and the area of neointimal proliferation calculated (in sq mm).

In control animals which received no treatment, or which were treated with the drug-free gel, there was extensive restenosis, characterized by symmetric neointimal formation along the entire length of the injured artery, narrowing the lumen by about 60%, resulting in an intima ratio of 1.4.

In animals treated with antisense c-myb oligonucleotides, there was minimal restenosis, minimal proliferative rim (less than 10% of the lumen) that was limited to the portion of the artery in direct contact with oligonucleotide, with an intima/media ratio of 0.09. This effect was most pronounced for animals treated with the antisense/Pluronic®. The intima/media ratio obtained using EVAc/antisense was about 0.45. However, the EVAc matrix contained 40 Hg of oligonucleotide, compared with 200 μg of oligonucleotide administered in the Pluronic gel, which may account for some of the difference.

Seven rats in each treatment group were subjected to balloon angioplasty, and the arterial walls treated as follows: with a drug free hydrogel (Pluronic™ 127 as described above), a hydrogel containing sense c-myb, a hydrogel containing antisense c-myb, and no treatment at all. Similar high levels of neointimal proliferation occurred in all animals except those treated with antisense c-myb, where the levels of proliferation were dramatically lower.

### Example 6

### Inhibition of PCNA Using Antisense Oligonucleotides

Using the same methodology as in Example 4, antisense for PCNA having the sequence:

Sequence ID No. 7:

GAT CAG GCG TGC CTC AAA,

was applied to SV-SMC cells in culture. Sense PCNA was used as a negative control; NMMHC-B was used as a positive (inhibitory) control.

There was no suppression of smooth muscle cell proliferation in the negative control; there was 52% suppression using antisense NMMHC-B and 58% suppression with antisense PCNA.

### Example 7

### In vivo Application of Antisense Oligonucleotides to Inhibit Smooth Muscle Cell Proliferation in Rabbits

New Zealand white rabbits (1-1.5 Kg) were anesthetized with a mixture of Ketamine and zylazine and carotid dissection was performed as described in Example 4. A 5F Swan-Ganz catheter was inserted and positioned in the descending aorta with fluoroscopic guidance. The Swan-Ganz catheter was exchanged over the wire for an angioplasty catheter with a 3.0 mm balloon. The common iliac artery was angioplastied 3 times at 100 PSI for 90 seconds each time. A Wolinsky catheter was introduced and loaded with oligonucleotide solution in a total volume of 5cc normal saline. Saline was injected as a control in a counterlateral iliac artery. The oligonucleotides were a mixture of antisense mouse c-myb and human NMMHC (200 μM of each), described above. The mixture was injected under 5 atmospheres of pressure over 60 seconds. Two rabbits were treated with antisense oligonucleotide.

The animals were sacrificed 4 weeks later and the arteries were processed as described in Example 5 for rat arteries.

The results indicated a 50% reduction is neointimal proliferation in rabbit arteries treated with antisense compared to saline alone.

### Example 8

### Inhibition of Proliferation of Baboon Smooth Muscle Cells Using Antisense Oligonucleotides

Using the same methodology as in Example 3, primary baboon smooth muscle cells (gift from Dr. Hawker, Emory University) were treated with antisense human myb and human NMNHC. The cells were allowed to grow for 72 hours after treatment with the oligonucleotides, then counted as described in Example 3. The results show that hNMMHC caused 65.5% growth suppression and c-myb caused 59.77% growth suppression in the baboon cells.

### Example 9

### Compositions for Use in the Prevention of Restenosis

It will be appreciated from the above teachings that antisense oligonucleotides to PCNA, NMMHC, c-myb and PDGFR-β and any mixture thereof can be made and used by themselves or in a suitable carrier. The above examples are therefore not restrictive.

Equivalents

One skilled in the art will recognize several equivalents, modifications, variations of the present method from the foregoing detailed description. Such equivalents, modifications and variations are intended be encompassed by the appended claims.

### REFERENCES

1. Clowes A W, Schwartz S M. Significance of quiescent smooth muscle migration in the injured rat carotid artery. Circ. Res. 1985; 56: 139-145

2. Lindner V, Reidy M A. Proliferation of smooth muscle cells after vascular injury is inhibited by an antibody against basic fibroblast growth factor. Proc. Natl. Acad. Sci USA. 1991; 88: 3739-3743.

3. Olson N E, Chao S. Lindner V, Reidy M A. Intimal smooth muscle cell proliferation after balloon catheter injury: the role of basic fibroblast growth factor. Am. J. Pathol. 1992; 140: 1017-1023.

4. Schwartz S M, Deblois D, O'Brien E R M. The intima: Soil for atherosclerosis and restenosis. Circ. Res. 1995; 77:445465.

5. Clowes A W, Clowes H M, Reidy M A. Kinetics of cellular proliferation after arterial injury III. Endothelial and smooth muscle growth factor in chronically denuded vessels. Lab. Invest. 1986; 54: 295-303.

6. Koyama N., Hart C E, Clowes A W. Different functions of the platelet-derived growth factor-α and -β receptors for the migration and proliferation of cultured baboons smooth muscle cells. Circ. Res. 1994; 75: 682-691.

7. Jawien A, Bowen-Pope D F, Lindner V, Schwartz S M, Clowes A W. Platelet-derived growth factor promotes smooth muscle migration and intimal thickening in a rat model of balloon angioplasty. J. Clin. Invest. 1992; 89: 507-511.

8. Ferns G A A, Raines E W, Sprugel K H, Motani A S, Reidy M A, Ross R. Inhibition of neointimal smooth muscle accumulation after angioplasty by antibody to PDGF. Science. 1991; 253:1129-1132.

9. Lau K-W, Sigwart U. Restenosis an accelerated arteriopathy: pathophysiology, preventive strategies and research horizons. In Molecular interventions and local drug delivery. Edelman E R, Levy R J editors. Saunders WB Company, Cambridge UK. 1995; 1-28.

10. Simons M, Edelman E R, DeKeyser J L, Langer R, Rosenberg R D. Antisense c-myb oligonucleotides inhibit intimal arterial smooth muscle cell accumulation in vivo. Nature. 1992; 359: 67-70.

11. Morishita R, Gibbons G H, Ellison K E, Nakajima M, Zhang L, Kaneda Y, Ogihara T, Dzau V J. Single intraluminal delivery of antisense cdc2 kinase and proliferating-cell nuclear antigen oligonucleotides results in chronic inhibition of neointimal hyperplasia. Proc. Natl. Acad. Sci. USA. 1993; 90: 8474-8478.

12. Morishita R, Gibbons G, Ellison K E, Nakajima M, von der Leyen H, Zhang L, Kaneda Y, Ogihara T. Dzau V. Intimal hyperplasia after vascular injury is inhibited by antisense cdk2 kinase oligonucleotides. J. Clin. Invest. 1994; 93:1458-1464.

13. Abe J-I, Zhou W, Taguchi J-I, Takuwa N. Miki K, Okazaki H, Kurokawa K, Kumada M, Takuwa Y. Suppression of neointimal smooth muscle cell accumulation in vivo by antisense cdc2 and cdk2 oligonucleotides in rat carotid artery. Biochem. Biophys. Res. Comm. 1994; 198: 16-24.

14. Simons M, Edelman E R, Rosenberg R D. Antisense proliferating cell nuclear antigen oligonucleotides inhibit intimal hyperplasia in a rat carotid artery injury model. J. Clin. Invest. 1994; 93: 2351-2356.

15. Bennett, M R, Anglin S, McEwan J R, Jagoe R, Newby A C, Evan G I. Inhibition of vascular smooth muscle cells proliferation in vitro and in vivo by c-myc antisense oligodeoxynucleotides. J. Clin. Invest. 1994; 93: 820-828.

16. Shi Y. Fard A, Galeo A, Hutchinson H G, Vermani P. Dodge G R, Hall D J, Shaheen F, Zalewski A. Transcatheter delivery of c-myc antisense oligomers reduces neointimal formation in a porcine model of coronary artery balloon injury. Circulation. 1994; 90: 944-951.

17. Edelman E R, Simons M, Sirois M G, Rosenberg R D. C-myc in vasculoproliferative disease. Circ. Res. 1995; 76:176-182.

18. Crooke R. In vitro toxicology and pharmacokinetics of antisense oligonucleotides. Anticancer Drug Des. 1991; 6: B09646.

19. Loke S, Stein C, Zhang X, Mori K, Nakanishi M, Subashinge C, Cohen J, Neckers L. Characterization of oligonucleotide transport into living cells. Proc. Natl. Acad. Sci. USA. 1989; 86: 3474-3478.

20. Yakubov L A, Deeva E A, Zarytova V F, Ivanova E M, Ryte A S, Yurchenko L V, Vlassov W. Mechanism of oligonucleotide uptake by cells: involvement of specific receptors? Proc. Natl. Acad. Sci. USA. 1989; 86: 6454-6458.

21. Wagner R, Nishikura K. Cell cycle expression of RNA duplex unwinding activity in cells. Mol. Cell Biol. 1988; 8: 770-777.

22. Raines E W, Bowen-Pope D F, Ross R. Platelet-derived growth factor. In Peptide growth factors and their receptors 1. M. B. Sporn and A. B. Roberts, editors. Springer-Verlag, New York. 1991; 173-262.

23. MajesKy M W, Reidy M A, Bowen-Pope D F, Hart C E, Wilcox J N, Schwartz S M. PDGF ligand and receptor gene expression during repair of arterial injury. J. Cell Biol. 1990; 111: 21492158.

24. Langer R. Brown L, Edelman E R. Controlled release and magnetically modulated release systems for macromolecules. Drug and enzyme targeting. Methods Enzymol. 1985; 112: 399423.

25. Edelman E R, Adams D A, Karnovsky M J. Effect of controlled adventitial heparin delivery on smooth muscle cell proliferation following endothelial injury. Proc. Natl. Acad. Sci. USA. 1990; 87: 3773-3777.

26. Rhine W D, Sukhatme S, Hsieh D S T, Langer R. A new approach to achieve zero-order release kinetics from diffusion-controlled polymer matrix systems. In Controlled Release of Bioactive Materials. R. Baker, editor. Academic Press, New York. 1980; 177-187.

27. Ross R. The smooth muscle cell: II. Growth of smooth muscle cell in culture and formation of elastic fibers. J. Cell Biol. 1971; 50: 172-186.

28. Simons M, Rosenberg R D. Antisense nonmuscle myosin heavy chain and c-myb oligonucleotides suppress smooth muscle cell proliferation in vitro. Circ. Res. 1992; 70: 835-843.

29. Ebbecke M, Unterberg C, Buchwald A, Stohr S, Wiegand V. Anti-proliferative effects of a c-myc antisense oligonucleotide on human arterial smooth muscle cells. Basic Res. Cardiol. 1992; 87: 585-591.

30. Biro S, Fu Y M, Yu Z X, Epstein S E. Inhibitory effects of antisense oligodeoxynucleotides targeting c-myc mRNA on smooth muscle cell proliferation and migration. Proc. Natl. Acad. Sci. USA. 1993: 90 654-658.

31. Shi Y, Hutchinson H G, Hall D J, Zalewski A. Downregulation of c-myc expression by antisense oligonucleotides inhibits proliferation of human smooth muscle cells. Circulation. 1993; 88: 1190-1195.

32. Nabel E G, Yang Z, Liptay S, Sang H, Gordon D, Haudenschild C C, Nabel G J. Recombinant platelet-derived growth factor B gene expression in porcine arteries induces intimal hyperplasia in vivo. J. Clin. Invest. 1993; 91: 1822-1829.

33. Baumgartner H R, Platelet interaction with vascular structures. Thromb Diath. Haemorrh. Suppl. 1972; 51: 161-176.

34. Heldin C H, Watson A, Westermark B. Partial purification and characterization of platelet factors stimulating the multiplication of human glial cells. Exp. Cell Res. 1977; 109: 429437.

35. Assoian R K, Grotendorst G R, Miller D M, Spom M B. Cellular transformation by coordinate action of three peptide growth factors from human platelets. Nature. 1984; 309: 804806.

36. Hwang D L, Latus L J, Lev-Ran A. Effects of platelet-contained growth factors (PDGF, EGF, IGF-1, and TGF-P) on DNA synthesis in porcine aortic smooth muscle cells in culture. Exp. Cell Res. 1992; 200: 358360.

37. Burgess T L, Fisher E F, Ross S L, Bready J V, Qian Y-X, Bayewitch L A, Cohen A M, Herrara C J, Hu SS-F, Kramer T B, Lott F D, Martin F H, Pierce G F, Simonet L, Farrell C L. The antiproliferative activity of c-myb and c-myc antisense oligonucleotides in smooth muscle cells is caused by a nonantisense mechanism. Proc. Natl. Acad. USA. 1995; 92: 40514055.

38. Guvakova M A, Yakubov I A, Vlodavsky I, Tonkinson J L, Stein C A. Phosphorothioate oligodeoxynucleotides bind to basic fibroblast growth factor, inhibit its binding to cell surface receptors, and remove it from low affinity binding sites on extracellular matrix. J. Biol. Chem. 1995; 270: 2620-2627.

39. Stein C A. Does antisense exist? Nature Med. 1995; 1: 1119-1121.

40. Wang A, Creasy A, Lardner M, Lin L, Strickler J, Van Arsdell J A Yamamoto R, Mark D. Molecular cloning of the complementary DNA for human tumor necrosis factor. Science. 1985; 228: 149-154.

41. Holt J T, Redner R L, Nienhuis A W. An oligomer complementary to c-myc mRNA inhibits proliferation of HL-60 promyelocytic cells and induces differentiation. Mol. Cell Biol. 1988; 8: 963-973.

42. Paules R S, Buccione R, Moschel R C, Vande Woude G F, Eppig J J. Mouse mos protooncogene product is present and functions during oogenesis. Proc. Natl. Acad. Sci. USA. 1989; 86: 5395-5399.

43. Daaka Y, Wickstrom E. Target dependence of antisense oligodeoxynucleotide inhibition of c-Ha-ras p21 expression and focus formation in T24-transformed NIH3T3 cells. Oncogene Res. 1990; 5 267-275.

44. Liebhaber S, Russell J E, Cash F, Eshelman S S. Intramolecular duplexes in eukaryotic mRNA suppress translation in a position-dependent manner. J. Cell. Biochem. 1991; 15D: CD007 (Abstract).

45. Speir E, Epstein S E. Inhibition of smooth muscle cell proliferation by an antisense oligodeoxynucleotide targeting the messenger RNA encoding proliferating cell nuclear antigen. Circulation. 1992; 86: 538-547.

46. Wickstrom E, Simonet W, Medlock, Ruiz-Robles I. Complementary oligonucleotide probe of vesicular stomatitis virus matrix protein mRNA translation. Biophys. J. 1986; 49: 15-17.

47. Kozak M. Influences of mRNA secondary structure on initiation by eucaryotic ribosomes. Proc. Natl. Acad. Sci USA. 1988; 85; 2850-2854.

48. Jaroszewski J W, Kaplan O, Syi J L, Sehested M. Faustino P J, Cohen J S. Concerning antisense inhibition of the multidrug resistance gene. Cancer Commun. 1990; 2; 287-294.

49. Tanizawa S, Ueda M, van der Loos C M, et al. Expression of platelet derived growth factor B chain and, receptor in human coronary arteries after percutaneous transluminal coronary angioplasty: an immunohistochemical study. Heart. 1996;75.549-556.

50. Bilder G, Wentz T, Leadley R, et al. Restenosis following angioplasty in the swine coronary artery is inhibited by an orally active PDGF-receptor tyrosine kinase inhibitor, RPR01511A. Circulation. 1999;99: 32!2-3299.

51. Banai S. Wolf Y. Golomg G, et al. PDGF-receptor tyrosine kinase blocker AG1295 selectively attenuates smooth muscle cell growth in vitro and reduces neointimal formation after balloon angioplasty in swine. Circulation. 1998;97: 1960-1969.

52. Hart C E, Kraiss L W, Vergel S, et al. PDGFβ receptor blockade inhibits intimal hyperplasia in the baboon. Circulation. 1999;99:564-569.

What is claimed is:

1. A method of inhibiting translation or transcription of a target nucleic acid sequence encoding a protein involved in smooth muscle migration and/or proliferation within a blood vessel of a mammal suffering of vascular injury, which comprises the step of: directly depositing onto a surface or within the blood vessel at least one oligonucleotide complementary to the target sequence, in an amount sufficient to penetrate cells of the blood vessel, to hybridize with said target nucleic acid, and to inhibit intracellular translation or transcription of said target sequence, said protein comprising platelet-derived growth factor P-receptor subunit (PDGFR-β).

2. The method of claim 1 which results in prevention of restenosis.

3. The method of claim 1 wherein the oligonucleotide is in a physiologically compatible solution and wherein it is applied by injection.

4. The method of claim 3 wherein the solution is applied to the tissue using an infusion pump, stent or catheter.

5. The method of claim 1 wherein said at least one oligonucleotide further comprises an antisense sequence complementary to the sequence of a gene selected from the group consisting of c-myb, NMMHC and PCNA.

6. The method of claim 1 wherein said oligonucleotide sequence comprises about 14 to 38 nucleotides bases.

7. The method of claim 1 where said at least one oligonucleotide is treated to render it resistant to degradation or extension by intracellular enzymes.

8. The method of claim 7 wherein the treatment comprises substituting at least one backbone phosphodiester linkage of the oligonucleotide with a linkage selected from the group consisting of phosphorothioate, methylphosphonate, sulfone, sulfate, ketyl, phosphorodithioate, various phosphoramidate, phosphate ester, bridged phosphorothioate and bridged phosphoramidate linkages.

9. The method of claim 7 wherein the treatment comprises capping a 3′-nucleotide with a structure resistant to addition of nucleotides.

10. The method of claim 1 wherein said at least one oligonucleotide is delivered to the blood vessel in a concentration of between approximately 30 and 3000 μg oligonucleotide per square centimeter of tissue surface area.

11. The method of claim 1 wherein the target nucleic acid sequence comprises a mRNA.

12. The method of claim 11 wherein the oligonucleotide is incorporated into a carrier.

13. The method of claim 12 wherein the carrier comprises an implantable matrix.

14. The method of claim 12 wherein the carrier comprises a hydrogel.

15. The method of claim 14 wherein the hydrogel comprises a material which is liquid at a temperature below 37° C.

16. The method of claim 15 wherein the hydrogel material comprises a polyoxethylene oxide and polypropylene oxide copolymer.

17. The method of claim 16 wherein the copolymer comprises from about 10 to about 80% by weight polyethylene oxide and form about 20 to about 90% polypropylene oxide.

18. The method of claim 17 wherein the polymer comprises about 70% by weight polyethylene oxide and about 30% by weight polypropylene oxide.

19. The method of claim 1 wherein the oligonucleotide is deposited extravascularly.

20. The method of claim 1 wherein said oligonucleotide is deposited onto or beneath an adventitial surface of the blood vessels.
